SEQUENCE LISTING (5 sequences)

SEQ ID NO: 1 - DNA, 868 bp, Homo sapiens
ataagaatgcggccgcatgaccccgcagcttctcctggcccttgtcctctgggccagctgcccgccccgcagtggaaggaaagggcccccagcagctctgacactgccccgggtgcaatgccgagcctctcggtacccgatcgccgtggattgctcctggaccctgccgcatgaccccgcagcttctcctggcccttgtcctctgggccagctgcccgccctgcggtggaaggaaagggcccccagcagctctgacactgccccgggtgcaatgccgagcctctcggtacccgatcgccgtggattgctcctggaccctgccgcctgctccaaactccaccagccccgtgtccttcattgccacgtacaggctcggcatggctgcccggggccacagctggccttgcctgcagcagacgccaacgtccaccagctgcaccatcacggatgtccagctgttctccatggctccctacgtgctcaatgtcaccgccgtccgcccctggggctccagcagcagcttcgtgcctttcataacagagcacatcatcaagcccgaccctccagaaggcgtgcgcctaagccccctcgctgagcgccagctacaggtgcagtgggagcctcccgggtcctggcccttcccagagatcttctcactgaagtactggatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagtctccccgccactgccacaatgagcctgggcaagtagactagtcc

SEQ ID NO: 2 - protein, 192 aa, Homo sapiens [sequence omitted]

SEQ ID NO: 3 - protein, 192 aa, Homo sapiens [sequence omitted]

SEQ ID NO: 4 - protein, 229 aa, Homo sapiens [sequence omitted]

SEQ ID NO: 5 - protein, 14 aa, Homo sapiens
  misc_feature 13: Xaa may be any amino acid
CXXXXXXXXXXCXW

(19) United States
(12) Patent Application Publication
(10) Pub. No.: US 2002/0086982 A1
(43) Pub. Date: Jul. 4, 2002

(54) Novel EBI-3-ALT protein and nucleic acid molecules and uses therefor

(76) Inventor: Michael R. Bowman, Westwood, MA (US)

Correspondence Address: LAHIVE & COCKFIELD, 28 STATE STREET, BOSTON, MA, 02109, US

(21) Appl. No.: 09/921,994

(22) Filed: Aug. 3, 2001

Related U.S. Application Data
(60) Provisional application No. 60/223,285, filed on Aug. 3, 2000.

(51) Int. Cl. C07H 21/02; C07H 21/04; C07K 1/00; C07K 14/00; C07K 17/00

(52) U.S. Cl. 536/23.1; 530/350

(57) ABSTRACT

Novel EBI-3-alt polypeptides, proteins, and nucleic acid molecules are disclosed. In addition to isolated, full-length EBI-3-alt proteins, the invention further provides isolated EBI-3-alt fusion proteins, antigenic peptides and anti-EBI-3-alt antibodies. The invention also provides EBI-3-alt nucleic acid molecules, recombinant expression vectors containing a nucleic acid molecule of the invention, host cells into which the expression vectors have been introduced and non-human transgenic animals in which an EBI-3-alt gene has been introduced or disrupted. Diagnostic, screening and therapeutic methods utilizing compositions of the invention are also provided.

### RELATED APPLICATION

The present application claims priority to U.S. Provisional Patent Application Ser. No. 60/223,285, filed Aug. 3, 2000, entitled “Novel EBI-3-ALT Protein and Nucleic Acid Molecules and Uses Therefor”, the entire contents of which are expressly incorporated by reference.

### BACKGROUND OF THE INVENTION

One of the central paradigms of evolution is that genetic duplication and transposition events have been important sources of novel genes and gene functions. This view has been buttressed by the recent explosive increase in available biological sequence data that highlights the richness of the homologous relationships that exist between genes and proteins in most all organisms. It is now clear that protein sequences and their composite elements (e.g., domains, motifs, consensus sequences) have evolved into complex combinations and families through a variety of genetic rearrangement mechanisms. Some of these mechanisms can cause shuffling events that are dispersed throughout a whole genome, for example the duplication of an entire gene cluster (Kappen et al (1989) PNAS 86:54595463) or transposable elements which give rise to interspersed repeats (Smit (1996) Curr Op. Gen. Dev. 6:743-748). Other mechanisms cause more discrete rearrangements, for example, a single intergenic unequal crossover event in sister chromatids during meiosis will result in four simultaneous alterations: a deletion, a duplication, and two reciprocal recombinant genes (Jelesko et al (1999) PNAS 96:10302-10307). The combined impact of such disparate recombination events has been important in the evolution of complex loci such as HOX (Ruddle et al (1994) Annu. Rev. Genet. 28:423-442), amylase (Gumucio et al (1988) Mol. Cell. Biol. 8:1197-1205), and MHC (Hughs and Yeager (1997) BioEssays 19:777-786). It is clear that composite proteins comprising multiple portions of other proteins (“chimeras”) make up a large fraction of the protein complement of an organism (Henikoff et al (1997) Science 278:609-614; Gogarten and Olendzenski (1999) Curr. Opinion Genet. Dev. 9:630-636).

Consequently, novel chimeric genes and proteins that have been identified by their homologies to the genes from which their composite elements originated will have therapeutic potential. Such novel biological molecules may play important and/or unexpected roles in vital cellular processes.

### SUMMARY OF THE INVENTION

The present invention is based, at least in part, on the discovery of novel molecules having homology to EBI-3. These novel molecules are referred to herein as “EBI-3 alternative C-terminus” (also referred to herein as “EBI-3-alt”) nucleic acid and protein molecules and are useful as modulating agents in regulating a variety of cellular processes. Accordingly, in one aspect, this invention provides isolated nucleic acid molecules encoding EBI-3-alt proteins or biologically active portions thereof, as well as nucleic acid fragments suitable as primers or hybridization probes for the detection of EBI-3-alt-encoding nucleic acids.

In one embodiment, an EBI-3-alt nucleic acid molecule is 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9% or more homologous to the nucleotide sequence shown in SEQ ID NO:1 or SEQ ID NO:3, the nucleotide sequence of the DNA insert of the plasmid deposited with ATCC as Accession Number ______, or a complement thereof. In another embodiment, an isolated EBI-3-alt nucleic acid molecule encodes the amino acid sequence of human EBI-3-alt shown in SEQ ID NO:2.

In a preferred embodiment, an isolated EBI-3-alt nucleic acid molecule has the nucleotide sequence of SEQ ID NO:1, or a complement thereof. In another embodiment, an EBI-3-alt nucleic acid molecule further comprises nucleotides 1-16 of SEQ ID NO:1. In another embodiment, an EBI-3-alt nucleic acid molecule further comprises nucleotides 596-868 of SEQ ID NO:1. In yet another embodiment, an EBI-3-alt nucleic acid molecule comprises the nucleotide sequence of SEQ ID NO:3.

In yet another preferred embodiment, an isolated EBI-3-alt nucleic acid molecule has the nucleotide sequence of the DNA insert of the plasmid deposited with ATCC as Accession Number ______, or a complement thereof.

In another embodiment, an EBI-3-alt nucleic acid molecule includes a nucleotide sequence encoding a protein having an amino acid sequence sufficiently homologous to the amino acid sequence of SEQ ID NO:2. In yet another embodiment, an EBI-3-alt nucleic acid molecule includes a nucleotide sequence encoding a protein having an amino acid sequence at least 50%, 60%, 65%, 70%, 75%, 80%, 85%, 90%, 95%, 99% or more homologous to the amino acid sequence of SEQ ID NO:2. In a preferred embodiment, an EBI-3-alt nucleic acid molecule includes a nucleotide sequence encoding a protein having the amino acid sequence of SEQ ID NO:2.

In another embodiment, an isolated nucleic acid molecule of the present invention encodes an EBI-3-alt protein which includes an “N-terminal EBI-3-like domain”, and/or a “C-terminal unique domain”. In another embodiment, the EBI-3-alt nucleic acid molecule encodes an EBI-3-alt protein and is a naturally occurring nucleotide sequence. In yet another embodiment, an isolated nucleic acid molecule of the present invention encodes an EBI-3-alt protein and comprises a nucleotide sequence which hybridizes under stringent hybridization conditions to a nucleic acid molecule complementary to the nucleotide sequence of SEQ ID NO:1 or SEQ ID NO:3 or of the DNA insert of the plasmid deposited with ATCC as Accession Number ______.

Another embodiment of the invention features EBI-3-alt nucleic acid molecules that specifically detect EBI-3-alt nucleic acid molecules relative to nucleic acid molecules encoding non-EBI-3-alt proteins. For example, in one embodiment, an EBI-3-alt nucleic acid molecule hybridizes under stringent conditions to a nucleic acid which is complementary to a nucleic acid molecule comprising nucleotides 1-868 of SEQ ID NO:1. In another embodiment, the EBI-3-alt nucleic acid molecule is at least 50-100, 100-200, 200-300, 300-400, 400-500, 500-600, 600-700, 700-800, 800-860, or more nucleotides in length and hybridizes under stringent conditions to a nucleic acid which is complementary to a nucleic acid molecule comprising the nucleotide sequence shown in SEQ ID NO:1 or SEQ ID NO:3, the nucleotide sequence of the DNA insert of the plasmid deposited with ATCC as Accession Number ______, or a complement thereof.

Another embodiment the invention provides an isolated nucleic acid molecule which is antisense to the coding strand of an EBI-3-alt nucleic acid.

Another aspect of the invention provides a vector comprising an EBI-3-alt nucleic acid molecule. In certain embodiments, the vector is a recombinant expression vector. In another embodiment the invention provides a host cell containing a vector of the invention. The invention also provides a method for producing EBI-3-alt protein by culturing in a suitable medium, a host cell of the invention containing a recombinant expression vector such that EBI-3-alt protein is produced.

Another aspect of this invention features isolated or recombinant EBI-3-alt proteins and polypeptides. In one embodiment, an isolated EBI-3-alt protein has at least one of the following domains: an N-terminal EBI-3-like domain, a C-terminal unique domain.

In another embodiment, an isolated EBI-3-alt protein has an amino acid sequence sufficiently homologous to the amino acid sequence of SEQ ID NO:2. In a preferred embodiment, an EBI-3-alt protein has an amino acid sequence at least about 60% homologous to the amino acid sequence of SEQ ID NO:2. In another embodiment, an EBI-3-alt protein has the amino acid sequence of SEQ ID NO:2.

Another embodiment of the invention features an isolated EBI-3-alt protein which is encoded by a nucleic acid molecule having a nucleotide sequence at least about 60% homologous to a nucleotide sequence of SEQ ID NO:1 or SEQ ID NO:3, or the nucleotide sequence of the DNA insert of the plasmid deposited with ATCC as Accession Number ______, or a complement thereof. This invention also features an isolated EBI-3-alt protein which is encoded by a nucleic acid molecule having a nucleotide sequence which hybridizes under stringent hybridization conditions to a complement of a nucleic acid molecule comprising the nucleotide sequence of SEQ ID NO:1 or SEQ ID NO:3, the nucleotide sequence of the DNA insert of the plasmid deposited with ATCC as Accession Number ______, or a complement thereof. In another embodiment, the polypeptide is fragment of an EBI-3-alt polypeptide comprising the amino acid sequence of SEQ ID NO:2, or an amino acid sequence encoded by the DNA insert of the plasmid deposited with ATCC as Accession Number ______, where the fragment comprises at least 5-10 contiguous amino acids of SEQ ID NO:2 (e.g., comprises at least 10 contiguous amino acids of SEQ ID NO:2 from about amino acid 55-192), or the amino acid sequence encoded by the DNA insert of the plasmid deposited with ATCC as Accession Number ______.

The EBI-3-alt proteins of the present invention, or biologically active portions thereof, can be operatively linked to a non-EBI-3-alt polypeptide to form EBI-3-alt fusion proteins. The invention further features antibodies that specifically bind EBI-3-alt proteins (e.g., antibodies which bind to an antigenic determinant contained within amino acid residues 55-192 of SEQ ID NO:2), such as monoclonal or polyclonal antibodies. In addition, the EBI-3-alt proteins or biologically active portions thereof can be incorporated into pharmaceutical compositions, which optionally include pharmaceutically acceptable carriers.

In another aspect, the present invention provides a method for detecting EBI-3-alt expression in a biological sample by contacting the biological sample with an agent capable of detecting an EBI-3-alt nucleic acid molecule, protein or polypeptide such that the presence of EBI-3-alt nucleic acid molecule, protein or polypeptide is detected in the biological sample.

In another aspect, the present invention provides a method for detecting the presence of EBI-3-alt activity in a biological sample by contacting the biological sample with an agent capable of detecting an indicator of EBI-3-alt activity such that the presence of EBI-3-alt activity is detected in the biological sample.

In another aspect, the invention provides a method for modulating EBI-3-alt activity comprising contacting the cell with an agent that modulates EBI-3-alt activity such that EBI-3-alt activity in the cell is modulated. In one embodiment, the agent inhibits EBI-3-alt activity. In another embodiment, the agent stimulates EBI-3-alt activity. In one embodiment, the agent is an antibody that specifically binds to EBI-3-alt protein (e.g., an EBI-3-alt-specific antibody). In another embodiment, the agent modulates expression of EBI-3-alt by modulating transcription of an EBI-3-alt gene or translation of an EBI-3-alt mRNA. In yet another embodiment, the agent is a nucleic acid molecule having a nucleotide sequence that is antisense to the coding strand of the EBI-3-alt mRNA or the EBI-3-alt gene.

In one embodiment, the methods of the present invention are used to treat a subject having a disorder characterized by aberrant or unwanted EBI-3-alt protein or nucleic acid expression or activity by administering an agent which is an EBI-3-alt modulator to the subject. In one embodiment, the EBI-3-alt modulator is an EBI-3-alt protein. In another embodiment, the EBI-3-alt modulator is an EBI-3-alt nucleic acid molecule. In yet another embodiment, the EBI-3-alt modulator is a peptide, peptidomimetic, or other small molecule. In a preferred embodiment, the disorder characterized by aberrant EBI-3-alt protein or nucleic acid expression is an EBI-3-alt associated disorder.

The present invention also provides a diagnostic assay for identifying the presence or absence of a genetic alteration characterized by at least one of (i) aberrant modification or mutation of a gene encoding an EBI-3-alt protein; (ii) mis-regulation of said gene; and (iii) aberrant post-translational modification of an EBI-3-alt protein, wherein a wild-type form of said gene encodes a protein with an EBI-3-alt activity.

In another aspect the invention provides a method for identifying a compound that binds to or modulates the activity of an EBI-3-alt protein, by providing an indicator composition comprising an EBI-3-alt protein having EBI-3-alt activity, contacting the indicator composition with a test compound, and determining the effect of the test compound on EBI-3-alt activity in the indicator composition to identify a compound that modulates the activity of an EBI-3-alt protein.

Other features and advantages of the invention will be apparent from the following detailed description and claims.

### BRIEF DESCRIPTION OF THE DRAWINGS

FIG. 1 depicts the cDNA sequence of human EBI-3-alt. The nucleotide sequence corresponds to nucleic acids 1 to 868 of SEQ ID NO:1.

FIG. 2 depicts the amino acid sequence of EBI-3-alt, corresponding to amino acids 1-192 of SEQ ID NO:2.

FIG. 3 depicts a hydrophobicity plot of the amino acids sequence of EBI-3-alt.

FIG. 4 depicts a alignment between the amino acid sequences of human EBI-3-alt and human EBI-3 (GenBank accession number AAA93 193; SEQ ID NO:4).

### DETAILED DESCRIPTION OF THE INVENTION

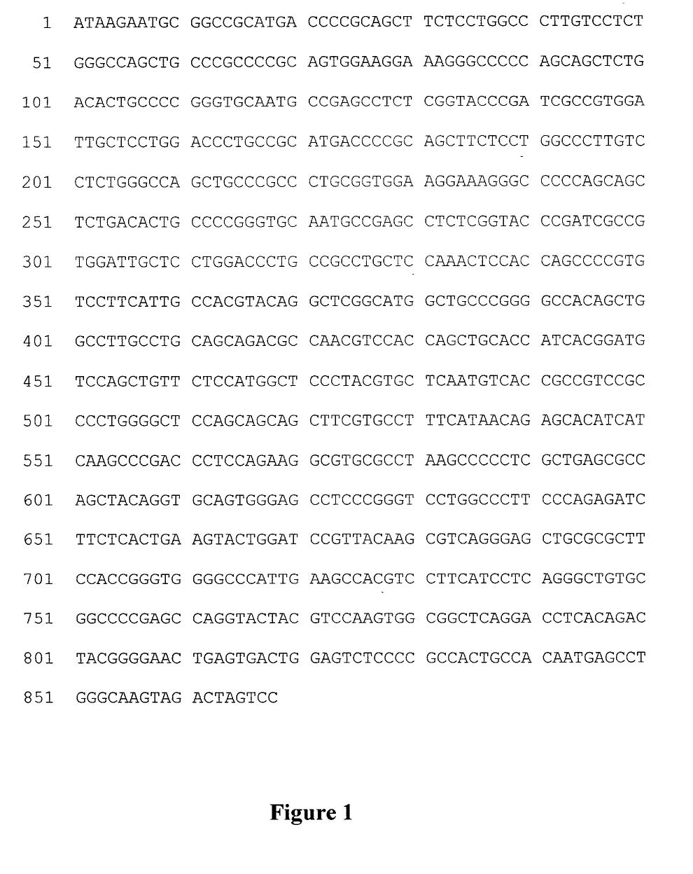

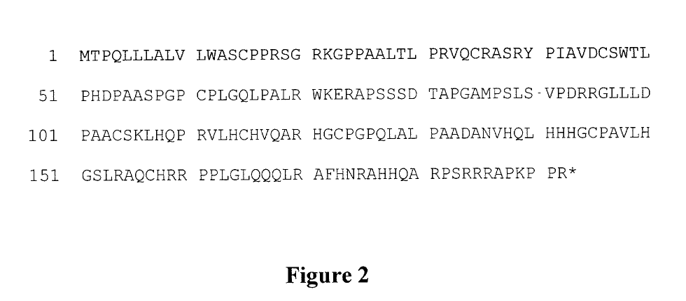

The present invention is based on the discovery of novel molecules, referred to herein as “EBI-3 alternative C-terminus” (also referred to herein as “EBI-3-alt”) protein and nucleic acid molecules, which comprise a family of molecules having certain conserved structural and functional features. The nucleotide sequence of a human EBI-3-alt nucleic acid molecule and the amino acid sequence of the predicted human EBI-3-alt protein molecule are depicted in FIGS. 1 and 2, respectively.

The term “family” when referring to the protein and nucleic acid molecules of the invention is intended to mean two or more protein or nucleic acid molecules having a common structural domain and having sufficient amino acid or nucleotide sequence homology as defined herein. Such family members can be naturally occurring and can be from either the same or different species. For example, a family can contain a first protein of human origin and an orthologue of that protein of murine origin, as well as a second, distinct protein of human origin (e.g., a homologue) and a murine orthologue of that protein. Members of a family may also have common functional characteristics. An exemplary family of the present invention is the EBI-3-alt protein family.

In another embodiment, an EBI-3-alt family member is identified based on the presence of an “N-terminal EBI-3-like domain” in the protein or corresponding nucleic acid molecule. As used herein, the term “N-terminal EBI-3-like domain” includes a protein domain which is about 30-80, preferably about 40-70, most preferably about 50-60 amino acid residues in length and has a primary structure that is 90%, 91%, 92%, 93%, 94%, 95%, 96%, 97%, 98%, 99%, or more identical to a portion of the EBI-3 protein. In a preferred embodiment, the portion of the EBI-3 protein to which the N-terminal EBI-3-like domain corresponds is the N-terminal portion of the human EBI-3 protein (e.g., from about amino acid position 1 to about amino acid position 54 of human EBI-3; SEQ ID NO:4). In another embodiment, an “N-terminal EBI-3-like domain” contains at least one “receptor_cytokines_{—}1” signature sequence. As used herein, a “receptor_cytokines_{—}1” signature sequence is a sequence routinely found towards the N-terminal portion of the extracellular domain of a number of receptors for lymphokines, hematopoeitic growth factors, and hormone related molecules. The cytokine receptor signature sequence has the consensus pattern C-[LVFYR]-x(7,8)-[STIVDN]-C-x-W (SEQ ID NO:5), wherein the two conserved cysteine residues can be linked by a disulfide bond. The signature sequence has been assigned the name “receptor_cytokines_{—}1” and the ProSite accession number PS00241 (http://www.expasy.ch/prosite). A “receptor_cytokines_{—}1” signature sequence can be found in the N-terminal EBI-3-like domain of the human EBI-3-alt protein from about amino acids 35-48 (SEQ ID NO:2). In addition, the receptor_cytokines_{—}1 signature sequence of the human EBI-3-alt protein contains the pair of conserved cysteine residues described above (cys 35 and cys 46 of SEQ ID NO:2).

An N-terminal EBI-3-like domain can be identified in a protein by its homology to amino acid residues 1-54 of SEQ ID NO:2 and/or by the presence of one or more receptor_cytokines_{—}1 signature sequences. An N-terminal EBI-3-like domain can be found in the EBI-3-alt protein (SEQ ID NO:2), for example, from amino acids 1 to 54.

The consensus sequence described herein has been described according to standard Prosite Signature designation (e.g., all amino acids are indicated according to their universal single letter designation; X designates any amino acid; X(n) designates any n amino acids, e.g., X (2) designates any 2 amino acids; [LIVM] indicates any one of the amino acids appearing within the brackets, e.g., any one of L, I, V, or M, in the alternative, any one of Leu, Ile, Val, or Met.); and {LIVM} indicates any amino acid except the amino acids appearing within the brackets, e.g., not L, not I, not V, and not M.

In another embodiment, an EBI-3-alt family member is identified based on the presence of an “C-terminal unique domain” in the protein or corresponding nucleic acid molecule. As used herein, the term “C-terminal unique domain” includes a protein domain which is about 80-200, more preferably about 100-180, or more preferably 120-160, or most preferably 130-150 amino acid residues in length, and has a primary structure that is 50%, 60%, 70%, 80%, 90%, 95%, 99%, 99.5% or more identical to a C-terminal portion of an EBI-3-alt protein. In a preferred embodiment, the C-terminal portion of an EBI-3-alt protein to which the “C-terminal unique domain” corresponds is the portion from about amino acid position 55 to the C-terminal amino acid (position 192) of the human EBI-3-alt protein (SEQ ID NO:2).

A C-terminal unique domain can be identified in a protein by its homology to amino acids 52-192 of SEQ ID NO:2. A C-terminal unique domain can be found in the EBI-3-alt protein (SEQ ID NO:2), for example, from amino acids 55-192.

Preferred EBI-3-alt molecules of the present invention have an amino acid sequence sufficiently homologous to the amino acid sequence of SEQ ID NO:2. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains and/or a common functional activity. For example, amino acid or nucleotide sequences which share common structural domains have at least about 60-65% homology, preferably 65-75% homology, more preferably 75-85%, even more preferably 85-95%, and most preferably 95-99.9% homology across the amino acid sequences of the domains and contain at least one, preferably two, and more preferably three or more structural domains, are defined herein as sufficiently homologous. Furthermore, amino acid or nucleotide sequences that share at least 60-65%, preferably 65-75%, more preferably 75-85%, and even more preferably 85-95%, most preferably 95-99.9% homology and share a common functional activity are defined herein as sufficiently homologous.

As used interchangeably herein an “EBI-3-alt activity”, “biological activity of EBI-3-alt” or “functional activity of EBI-3-alt”, refers to an activity exerted by an EBI-3-alt protein, polypeptide or nucleic acid molecule on an EBI-3-alt responsive cell as determined in vivo, or in vitro, according to standard techniques. In one embodiment, an EBI-3-alt activity is a direct activity, such as an association with a cell-surface protein (e.g., an EBI-3-alt receptor or EBI-3-alt binding protein). In another embodiment, an EBI-3-alt activity is an indirect activity, such as the modulation of the activity of a second protein (e.g. a cellular signaling molecule) mediated by interaction of the EBI-3-alt protein with a cell surface protein. In a preferred embodiment, an EBI-3-alt activity is at least one or more of the following activities: (i) interaction of an EBI-3-alt protein with a non-EBI-3-alt protein molecule (e.g., an EBI-3-alt receptor or a heteromeric multimer partner); (ii) interaction of an EBI-3-alt protein with one or more other EBI-3-alt protein molecules (e.g., a homomeric multimer partner); (iii) complex formation between an EBI-3-alt protein and an EBI-3-alt receptor; (iv) complex formation between an EBI-3-alt protein and non-EBI-3-alt receptor; (v) modulation of signal transduction pathways; (vi) modulation of a cellular response; (vii) modulation of levels of a EBI-3-alt ligand or metabolic product; (viii) modulation of a cellular response to an EBI-3-alt ligand; and (ix) modulation of cellular homeostasis.

In yet another preferred embodiment, an EBI-3-alt activity is at least one or more of the following activities: (i) activation of an EBI-3-alt-dependent signal transduction pathway; (ii) modulation of proliferation, development or differentiation of an EBI-3-alt-expressing cell or EBI-3-alt-receptor-expressing cell; (iii) modulation of the proliferation, development or differentiation of a non-EBI-3-alt-expressing cell or non-EBI-3-alt-receptor-expressing cell; (iv) modulation of the homeostasis of an EBI-3-alt-expressing cell; and (v) modulation of the homeostasis of a non-EBI-3-alt-expressing cell; and (vi) modulation of levels of EBI-3-alt ligands or metabolites.

The present invention is based, at least in part, on the discovery of a novel protein (the human EBI-3-alt protein having the amino acid sequence of SEQ ID NO:2) which was isolated from a human placental cDNA library and has local homology to at least a portion of EBI-3.

Accordingly, another embodiment of the invention features isolated EBI-3-alt proteins and polypeptides having an EBI-3-alt activity. Preferred EBI-3-alt proteins have one or more of the following domains: an N-terminal EBI-3-like domain, C-terminal unique domain, and have an EBI-3-alt activity. In a preferred embodiment, the EBI-3-alt protein has at least at least an N-terminal EBI-3-like domain, C-terminal unique domain, an EBI-3-alt activity, and an amino acid sequence sufficiently homologous to an amino acid sequence of SEQ ID NO:2.

In a particularly preferred embodiment, the EBI-3-alt protein and nucleic acid molecules of the present invention are human EBI-3-alt molecules. A human EBI-3-alt cDNA molecule was obtained from a human placental cDNA library as described in Example 1. The nucleotide sequence of the isolated human EBI-3-alt cDNA and the predicted amino acid sequence of the human EBI-3-alt protein are shown in FIG. 1 and 2 and in SEQ ID NOS:1 and 2, respectively. In addition, the nucleotide sequence corresponding to the coding region of the human EBI-3-alt cDNA is represented as SEQ ID NO:3.

The human EBI-3-alt I cDNA set forth in SEQ ID NO:1, is approximately 868 nucleotides in length and encodes a protein which is approximately 192 amino acid residues in length (SEQ ID NO:2) which is shown in FIG. 2. The human EBI-3-alt protein contains an N-terminal EBI-3-like domain and/or a C-terminal unique domain, as defined herein. An EBI-3-alt N-terminal EBI-3-like domain can be found at least, for example, from about amino acids 1-54 of SEQ ID NO:2. An EBI-3-alt C-terminal unique domain can be found at least, for example, from about amino acids 55-192 of SEQ ID NO:2.

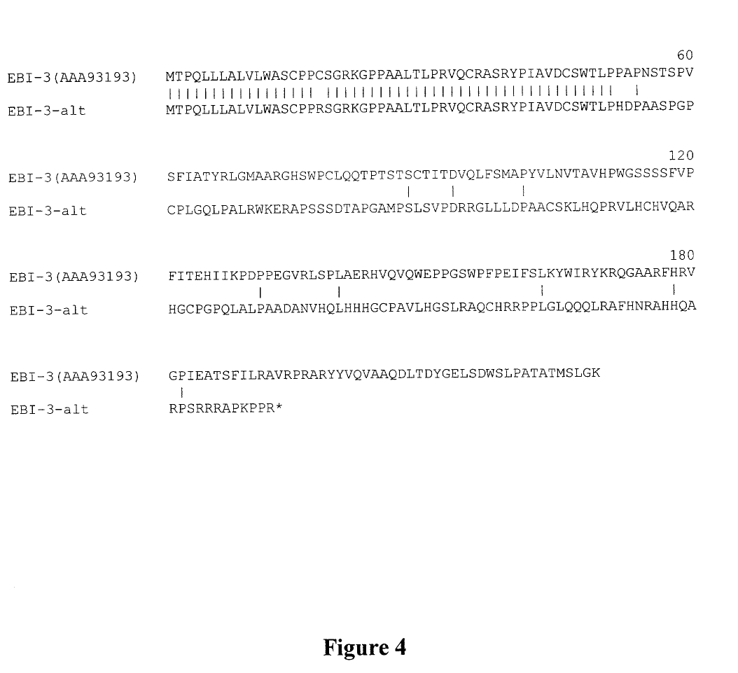

An alignment of the amino acid sequences of human EBI-3-alt and human EBI-3 (Genbank™ Accession No. AAA93193), is shown in FIG. 4.

A plasmid containing the nucleotide sequence encoding human EBI-3-alt was deposited with the American Type Culture Collection (ATCC), 10801 University Boulevard, Manassas, Va. 20110-2209, USA, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The human EBI-3-alt gene, which is approximately 868 nucleotides in length, encodes a protein having a molecular weight of approximately 21.1 kD and which is approximately 192 amino acid residues in length.

Various aspects of the invention are described in further detail in the following subsections:

I. Isolated Nucleic Acid Molecules

One aspect of the invention pertains to isolated nucleic acid molecules that encode EBI-3-alt proteins or biologically active portions thereof, as well as nucleic acid fragments sufficient for use as hybridization probes to identify EBI-3-alt-encoding nucleic acids (e.g., EBI-3-alt mRNA) and fragments for use as PCR primers for the amplification or mutation of EBI-3-alt nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated EBI-3-alt nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1 or SEQ ID NO:3, the nucleotide sequence of the DNA insert of the plasmid deposited with ATCC as Accession Number ______, or a portion thereof, can be isolated using standard molecular biology techniques and the sequence information provided herein. Using all or portion of the nucleic acid sequence of SEQ ID NO:1 or SEQ ID NO:3, or the nucleotide sequence of the DNA insert of the plasmid deposited with ATCC as Accession Number ______, as a hybridization probe, EBI-3-alt nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 or SEQ ID NO:3, or the nucleotide sequence of the DNA insert of the plasmid deposited with ATCC as Accession Number ______, can be isolated by the polymerase chain reaction (PCR) using synthetic oligonucleotide primers designed based upon the sequence of SEQ ID NO:1 or SEQ ID NO:3, or the nucleotide sequence of the DNA insert of the plasmid deposited with ATCC as Accession Number ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to EBI-3-alt nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1. The sequence of SEQ ID NO:1 corresponds to a human EBI-3-alt cDNA. This cDNA comprises sequences encoding a human EBI-3-alt protein (i.e., “the coding region”, from nucleotides 17-595), as well as 5′ untranslated sequences (nucleotides 1-16) and 3′ untranslated sequences (nucleotides 596-868). Alternatively, the nucleic acid molecule can comprise only the coding region of SEQ ID NO:1 (e.g., nucleotides 17-595).

In another preferred embodiment, an isolated nucleic acid molecule of the invention comprises the nucleotide sequence shown in SEQ ID NO:3. The sequence of SEQ ID NO:3 corresponds to the human EBI-3-alt cDNA. This cDNA comprises sequences encoding the human EBI-3-alt protein (i.e., “the coding region”, from nucleotides 1 to 193 of SEQ ID NO:3).

In yet another preferred embodiment, an isolated nucleic acid molecule of the invention comprises the nucleotide sequence of the DNA insert of the plasmid deposited with ATCC as Accession Number ______. A plasmid containing the full length nucleotide sequence encoding EBI-3-alt was deposited with the American Type Culture Collection (ATCC), Rockville, Md., and assigned Accession Number ______.

In another preferred embodiment, an isolated nucleic acid molecule of the invention comprises a nucleic acid molecule which is a complement of the nucleotide sequence shown in SEQ ID NO:1 or SEQ ID NO:3, the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:1 or SEQ ID NO:3, or the nucleotide sequence of the DNA insert of the plasmid deposited with ATCC as Accession Number ______ is one which is sufficiently complementary to the nucleotide sequence shown in SEQ ID NO:1 or SEQ ID NO:3, or the nucleotide sequence of the DNA insert of the plasmid deposited with ATCC as Accession Number ______ such that it can hybridize to a complement of the nucleotide sequence shown in SEQ ID NO:1 or SEQ ID NO:3, or the nucleotide sequence of the DNA insert of the plasmid deposited with ATCC as Accession Number ______, thereby forming a stable duplex.

In still another preferred embodiment, an isolated nucleic acid molecule of the present invention comprises a nucleotide sequence which is at least about 60-65%, preferably at least about 65-75%, more preferable at least about 75-85%, and even more preferably at least about 85-95%, most preferably 95%-99.9% or more homologous to the nucleotide sequence shown in SEQ ID NO:1 or SEQ ID NO:3, the nucleotide sequence of the DNA insert of the plasmid deposited with ATCC as Accession Number ______, or a portion of any of these nucleotide sequences. In still another preferred embodiment, an isolated nucleic acid molecule of the present invention comprises a nucleotide sequence which is at least about 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9% or more homologous to the nucleotide sequence shown in SEQ ID NO:1 or SEQ ID NO:3, the nucleotide sequence of the DNA insert of the plasmid deposited with ATCC as Accession Number ______, or a portion of any of these nucleotide sequences.

Moreover, the nucleic acid molecule of the invention can comprise only a portion of the nucleic acid sequence of SEQ ID NO:1 or SEQ ID NO:3, or the nucleotide sequence of the DNA insert of the plasmid deposited with ATCC as Accession Number ______, for example a fragment which can be used as a probe or primer or a fragment encoding a biologically active portion of EBI-3-alt. The nucleotide sequence determined from the cloning of the human EBI-3-alt gene allows for the generation of probes and primers designed for use in identifying and/or cloning EBI-3-alt homologues in other cell types, e.g., from other tissues, as well as EBI-3-alt homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of SEQ ID NO:1 or SEQ ID NO:3, or the DNA insert of the plasmid deposited with ATCC as Accession Number ______ sense, or an anti-sense sequence of SEQ ID NO:1 or SEQ ID NO:3, or the DNA insert of the plasmid deposited with ATCC as Accession Number ______, or of a naturally occurring mutant of SEQ ID NO:1 or SEQ ID NO:3, or the DNA insert of the plasmid deposited with ATCC as Accession Number ______.

Probes based on the human EBI-3-alt nucleotide sequence can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an EBI-3-alt protein, such as by measuring a level of an EBI-3-alt-encoding nucleic acid in a sample of cells from a subject e.g., detecting EBI-3-alt mRNA levels or determining whether a genomic EBI-3-alt gene has been mutated or deleted.

A nucleic acid fragment encoding a “biologically active portion of EBI-3-alt” can be prepared by isolating a portion of SEQ ID NO:1 or SEQ ID NO:3, or the DNA insert of the plasmid deposited with ATCC as Accession Number ______ which encodes a polypeptide having an EBI-3-alt biological activity (the biological activities of the EBI-3-alt proteins have previously been described), expressing the encoded portion of EBI-3-alt protein (e.g., by recombinant expression in vitro) and assessing the activity of the encoded portion of EBI-3-alt.

The invention further encompasses nucleic acid molecules that differ from the nucleotide sequence shown in SEQ ID NO:1 or SEQ ID NO:3 and from the nucleotide sequences of the DNA insert of the plasmid deposited with ATCC as Accession Number ______, or portions thereof, due to degeneracy of the genetic code and thus encode the same EBI-3-alt protein as that encoded by the nucleotide sequence shown in SEQ ID NO:1 or SEQ ID NO:3 or by the nucleotide sequence of the DNA insert of the plasmid deposited with ATCC as Accession Number ______. In another embodiment, an isolated nucleic acid molecule of the invention has a nucleotide sequence encoding a protein having an amino acid sequence shown in SEQ ID NO:2.

In addition to the human EBI-3-alt nucleotide sequences shown in SEQ ID NO:1 or SEQ ID NO:3, respectively, it will be appreciated by those skilled in the art that DNA sequence polymorphisms that lead to changes in the amino acid sequences of EBI-3-alt may exist within a population (e.g., the human population). Such genetic polymorphism in the EBI-3-alt gene may exist among individuals within a population due to natural allelic variation. As used herein, the terms “gene” and “recombinant gene” refer to nucleic acid molecules comprising an open reading frame encoding an EBI-3-alt protein, preferably a mammalian EBI-3-alt protein. Such natural allelic variations can typically result in 1-5% variance in the nucleotide sequence of the EBI-3-alt gene. Any and all such nucleotide variations and resulting amino acid polymorphisms in EBI-3-alt that are the result of natural allelic variation and that do not alter the functional activity of EBI-3-alt are intended to be within the scope of the invention.

Moreover, nucleic acid molecules encoding EBI-3-alt proteins from other species, and thus which have a nucleotide sequence which differs from the human sequences of SEQ ID NO:1 or SEQ ID NO:3, or the nucleotide sequence of the DNA insert of the plasmid deposited with ATCC as Accession Number ______ are intended to be within the scope of the invention. Nucleic acid molecules corresponding to natural allelic variants and homologues of the EBI-3-alt cDNA of the invention can be isolated based on their homology to the human EBI-3-alt nucleic acids disclosed herein using the human cDNA, or portions thereof, as a hybridization probe according to standard hybridization techniques under stringent hybridization conditions. For example, a viral EBI-3-alt cDNA can be isolated based on its homology to human EBI-3-alt.

Accordingly, in another embodiment, an isolated nucleic acid molecule of the invention is at least 15 nucleotides in length and hybridizes under stringent conditions to the nucleic acid molecule comprising the nucleotide sequence of SEQ ID NO:1 or SEQ ID NO:3, or the DNA insert of the plasmid deposited with ATCC as Accession Number ______. In other embodiment, the nucleic acid is at least 30, 50, 100, 250 or 500 nucleotides in length. As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions are hybridization in 4× sodium chloride/sodium citrate (SSC), at about 65-70° C. (or alternatively hybridization in 4× SSC plus 50% formamide at about 42-50° C.) followed by one or more washes in 1× SSC, at about 65-70° C. A preferred, non-limiting example of highly stringent hybridization conditions are hybridization in 1× SSC, at about 65-70° C. (or alternatively hybridization in 1× SSC plus 50% formamide at about 42-50° C.) followed by one or more washes in 0.3× SSC, at about 65-70° C. A preferred, non-limiting example of reduced stringency hybridization conditions are hybridization in 4× SSC, at about 50-60° C. (or alternatively hybridization in 6× SSC plus 50% formamide at about 40-45° C.) followed by one or more washes in 2× SSC, at about 50-60° C. Ranges intermediate to the above-recited values, e.g., at 65-70 ° C. or at 42-50° C. are also intended to be encompassed by the present invention. SSPE (1× SSPE is 0.1 5M NaCl, 10 mM NaH₂PO₄, and 1.25mM EDTA, pH 7.4) can be substituted for SSC (1× SSC is 0.1 5M NaCl and 15 mM sodium citrate) in the hybridization and wash buffers; washes are performed for 15 minutes after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995).

In addition to naturally-occurring allelic variants of the EBI-3-alt sequence that may exist in the population, the skilled artisan will further appreciate that changes can be introduced by mutation into the nucleotide sequence of SEQ ID NO:1 or SEQ ID NO:3, or the DNA insert of the plasmid deposited with ATCC as Accession Number ______, thereby leading to changes in the amino acid sequence of the encoded EBI-3-alt protein, without altering the functional ability of the EBI-3-alt protein. For example, nucleotide substitutions leading to amino acid substitutions at “non-essential” amino acid residues can be made in the sequence of SEQ ID NO:1 or SEQ ID NO:3, or the DNA insert of the plasmid deposited with ATCC as Accession Number ______. A “non-essential” amino acid residue is a residue that can be altered from the wild-type sequence of EBI-3-alt (e.g., the sequence of SEQ ID NO:2) without altering the biological activity, whereas an “essential” amino acid residue is required for biological activity. For example, amino acid residues that are conserved among the EBI-3-alt proteins of the present invention, are predicted to be particularly unamenable to alteration. Furthermore, amino acid residues that are conserved between EBI-3-alt proteins and other proteins having N-terminal EBI-3-like domains and/or C-terminal unique domains are not likely to be amenable to alteration.

Accordingly, another aspect of the invention pertains to nucleic acid molecules encoding EBI-3-alt proteins that contain changes in amino acid residues that are not essential for activity. Such EBI-3-alt proteins differ in amino acid sequence from SEQ ID NO:2, yet retain biological activity. In one embodiment, the isolated nucleic acid molecule comprises a nucleotide sequence encoding a protein, wherein the protein comprises an amino acid sequence at least about 60-65% homologous to the amino acid sequence of SEQ ID NO:2. Preferably, the protein encoded by the nucleic acid molecule is at least about 60-75% homologous to SEQ ID NO:2, more preferably at least about 75-85% homologous to SEQ ID NO:2, and even more preferably at least about 85-95% homologous to SEQ ID NO:2, and most preferably at least about 95-99% homologous to SEQ ID NO:2.

In an alternative embodiment, the isolated nucleic acid molecule comprises a nucleotide sequence encoding a protein, wherein the protein comprises an amino acid sequence at least about 50%, 60%, 65%, 70%, 75%, 80%, 85%, 90%, 95%, 99% or more homologous to SEQ ID NO:2.

An isolated nucleic acid molecule encoding an EBI-3-alt protein homologous to the protein of SEQ ID NO:2 can be created by introducing one or more nucleotide substitutions, additions or deletions into the nucleotide sequence of SEQ ID NO:1 or SEQ ID NO:3, or into the nucleotide sequence of the DNA insert of the plasmid deposited with ATCC as Accession Number ______, such that one or more amino acid substitutions, additions or deletions are introduced into the encoded protein. Mutations can be introduced into SEQ ID NO:1 or SEQ ID NO:3, or the DNA insert of the plasmid deposited with ATCC as Accession Number ______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in EBI-3-alt is preferably replaced with another amino acid residue from the same side chain family. Alternatively, in another embodiment, mutations can be introduced randomly along all or part of an EBI-3-alt coding sequence, such as by saturation mutagenesis, and the resultant mutants can be screened for EBI-3-alt biological activity to identify mutants that retain activity. Following mutagenesis of SEQ ID NO:1 or SEQ ID NO:3, or the DNA insert of the plasmid deposited with ATCC as Accession Number ______, the encoded protein can be expressed recombinantly and the activity of the protein can be determined.

In a preferred embodiment, a mutant EBI-3-alt protein can be assayed for (1) activation of an EBI-3-alt-dependent signal transduction pathway; (2) modulation of the development or differentiation or proliferation of an EBI-3-alt-expressing cell; (3) modulation of the development or differentiation or proliferation of a non-EBI-3-alt-expressing cell; (4) modulation of the homeostasis of an EBI-3-alt-expressing cell; (5) modulation of the homeostasis of a non-EBI-3-alt-expressing cell; (6) modulation of levels of an EBI-3-alt ligand or metabolite.

In addition to the nucleic acid molecules encoding EBI-3-alt proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire EBI-3-alt coding strand, or to only a portion thereof. In one embodiment, an antisense nucleic acid molecule is antisense to a “coding region” of the coding strand of a nucleotide sequence encoding EBI-3-alt. The term “coding region” refers to the region of the nucleotide sequence comprising codons which are translated into amino acid residues (e.g., the coding region of human EBI-3-alt corresponds to SEQ ID NO:3). In another embodiment, the antisense nucleic acid molecule is antisense to a “noncoding region” of the coding strand of a nucleotide sequence encoding EBI-3-alt. The term “noncoding region” refers to 5′ and 3′ sequences which flank the coding region that are not translated into amino acids (i.e., also referred to as 5′ and 3′ untranslated regions).

Given the coding strand sequences encoding EBI-3-alt disclosed herein (e.g., SEQ ID NO:3), antisense nucleic acids of the invention can be designed according to the rules of Watson and Crick base pairing. The antisense nucleic acid molecule can be complementary to the entire coding region of EBI-3-alt mRNA, but more preferably is an oligonucleotide which is antisense to only a portion of the coding or noncoding region of EBI-3-alt mRNA. For example, the antisense oligonucleotide can be complementary to the region surrounding the translation start site of EBI-3-alt mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an EBI-3-alt protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave EBI-3-alt mRNA transcripts to thereby inhibit translation of EBI-3-alt mRNA. A ribozyme having specificity for an EBI-3-alt-encoding nucleic acid can be designed based upon the nucleotide sequence of an EBI-3-alt cDNA disclosed herein (i.e., SEQ ID NO:1 or SEQ ID NO:3, or the DNA insert of the plasmid deposited with ATCC as Accession Number ______). For example, a derivative of a Tetrahymena L-19 IVS RNA can be constructed in which the nucleotide sequence of the active site is complementary to the nucleotide sequence to be cleaved in an EBI-3-alt-encoding mRNA. See, e.g., Cech et al. U.S. Pat. No. 4,987,071; and Cech et al. U.S. Pat. No. 5,116,742. Alternatively, EBI-3-alt mRNA can be used to select a catalytic RNA having a specific ribonuclease activity from a pool of RNA molecules. See, e.g., Bartel, D. and Szostak, J. W. (1993) Science 261:1411-1418.

Alternatively, EBI-3-alt gene expression can be inhibited by targeting nucleotide sequences complementary to the regulatory region of the EBI-3-alt (e.g., the EBI-3-alt promoter and/or enhancers) to form triple helical structures that prevent transcription of the EBI-3-alt gene in target cells. See generally, Helene, C. (1991) Anticancer Drug Des. 6(6):569-84; Helene, C. et al. (1992) Ann. N. Y Acad. Sci. 660:27-36; and Maher, L. J. (1992) Bioassays 14(12):807-15.

In preferred embodiments, the nucleic acids of EBI-3-alt can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. PNAS 93: 14670-675.

PNAs of EBI-3-alt can be used therapeutic and diagnostic applications. For example, PNAs can be used as antisense or antigene agents for sequence-specific modulation of gene expression by, e.g., inducing transcription or translation arrest or inhibiting replication. PNAs of EBI-3-alt can also be used, e.g., in the analysis of single base pair mutations in a gene by, e.g., PNA directed PCR clamping; as 'artificial restriction enzymes when used in combination with other enzymes, e.g., S1 nucleases (Hyrup B. (1996) supra); or as probes or primers for DNA sequence and hybridization (Hyrup B. et al. (1996) supra; Perry-O'Keefe supra).

In another embodiment, PNAs of EBI-3-alt can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of EBI-3-alt can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Research 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988). In addition, oligonucleotides can be modified with hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

In addition to the isolated nucleic acid molecules described above, the present invention also features isolated “genes” corresponding to the nucleic acid molecules of SEQ ID NO:1 or SEQ ID NO:3. As defined herein, the term “isolated gene” includes a nucleic acid molecule comprising introns and exons which is separated from genomic DNA sequences which naturally flank the gene (i.e., sequences located at the 5′ and 3′ ends of the naturally-occurring gene) in the genomic DNA of the organism from which the isolated gene is derived. As used herein, an isolated gene “corresponding to” the isolated nucleic acid molecules of SEQ ID NO:1 or SEQ ID NO:3 includes an isolated gene having a nucleotide sequence which includes fragments of contiguous nucleotides of the nucleic acid sequence of the isolated nucleic acid molecules of SEQ ID NO:1 or SEQ ID NO:3 (e.g., cDNA sequence corresponding to exons in the isolated gene), such fragments being interspersed with nucleotide sequence not set forth in SEQ ID NO:1 or SEQ ID NO:3 (e.g., cDNA sequence corresponding to introns in the isolated gene). In a preferred embodiment, the present invention features an isolated gene corresponding to SEQ ID NO:1 or SEQ ID NO:3. Accordingly, in another embodiment, the present invention features an isolated gene corresponding to SEQ ID NO:1 or SEQ ID NO:3.

II. Isolated EBI-3-alt Proteins and Anti-EBI-3-alt Antibodies

One aspect of the invention pertains to isolated EBI-3-alt proteins, and biologically active portions thereof, as well as polypeptide fragments suitable for use as immunogens to raise anti-EBI-3-alt antibodies. In one embodiment, native EBI-3-alt proteins can be isolated from cells or tissue sources by an appropriate purification scheme using standard protein purification techniques. In another embodiment, EBI-3-alt proteins are produced by recombinant DNA techniques. Alternative to recombinant expression, an EBI-3-alt protein or polypeptide can be synthesized chemically using standard peptide synthesis techniques.

An “isolated” or “purified” protein or biologically active portion thereof is substantially free of cellular material or other contaminating proteins from the cell or tissue source from which the EBI-3-alt protein is derived, or substantially free from chemical precursors or other chemicals when chemically synthesized. The language “substantially free of cellular material” includes preparations of EBI-3-alt protein in which the protein is separated from cellular components of the cells from which it is isolated or recombinantly produced. In one embodiment, the language “substantially free of cellular material” includes preparations of EBI-3-alt protein having less than about 30% (by dry weight) of non-EBI-3-alt protein (also referred to herein as a “contaminating protein”), more preferably less than about 20% of non-EBI-3-alt protein, still more preferably less than about 10% of non-EBI-3-alt protein, and most preferably less than about 5% non-EBI-3-alt protein. When the EBI-3-alt protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of EBI-3-alt protein in which the protein is separated from chemical precursors or other chemicals which are involved in the synthesis of the protein. In one embodiment, the language “substantially free of chemical precursors or other chemicals” includes preparations of EBI-3-alt protein having less than about 30% (by dry weight) of chemical precursors or non-EBI-3-alt chemicals, more preferably less than about 20% chemical precursors or non-EBI-3-alt chemicals, still more preferably less than about 10% chemical precursors or non-EBI-3-alt chemicals, and most preferably less than about 5% chemical precursors or non-EBI-3-alt chemicals.

Biologically active portions of an EBI-3-alt protein include peptides comprising amino acid sequences sufficiently homologous to or derived from the amino acid sequence of the EBI-3-alt protein, e.g., the amino acid sequence shown in SEQ ID NO:2, which include less amino acids than the full length EBI-3-alt proteins, and exhibit at least one activity of an EBI-3-alt protein. Typically, biologically active portions comprise a domain or motif with at least one activity of the EBI-3-alt protein. A biologically active portion of an EBI-3-alt protein can be a polypeptide which is, for example, 10, 25, 50, 100 or more amino acids in length.

In one embodiment, a biologically active portion of an EBI-3-alt protein comprises at least a N-terminal EBI-3-like domain. In yet another embodiment, a biologically active portion of an EBI-3-alt protein comprises at least a C-terminal unique domain.

It is to be understood that a preferred biologically active portion of an EBI-3-alt protein of the present invention may contain at least one of the above-identified structural domains. A more preferred biologically active portion of an EBI-3-alt protein may contain at least two of the above-identified structural domains. An even more preferred biologically active portion of an EBI-3-alt protein may contain at least three or more of the above-identified structural domains.

Moreover, other biologically active portions, in which other regions of the protein are deleted, can be prepared by recombinant techniques and evaluated for one or more of the functional activities of a native EBI-3-alt protein.

In a preferred embodiment, the EBI-3-alt protein has an amino acid sequence shown in SEQ ID NO:2. In other embodiments, the EBI-3-alt protein is substantially homologous to SEQ ID NO:2 and retains the functional activity of the protein of SEQ ID NO:2, yet differs in amino acid sequence due to natural allelic variation or mutagenesis, as described in detail in subsection I above. Accordingly, in another embodiment, the EBI-3-alt protein is a protein which comprises an amino acid sequence at least about 60% homologous to the amino acid sequence of SEQ ID NO:2 and retains the functional activity of the EBI-3-alt proteins of SEQ ID NO:2. Preferably, the protein is at least about 70% homologous to SEQ ID NO:2, more preferably at least about 80% homologous to SEQ ID NO:2, even more preferably at least about 90% homologous to SEQ ID NO:2, and most preferably at least about 95% or more homologous to SEQ ID NO:2.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the EBI-3-alt amino acid sequence of SEQ ID NO:2, having 192 amino acid residues, at least 58, preferably at least 77, more preferably at least 96, even more preferably at least 115, or even more preferably at least 134, more preferably 155, or most preferably 173 amino acids are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

The comparison of sequences and determination of percent homology between two sequences can be accomplished using a mathematical algorithm. A non-limiting example of a mathematical algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score =100, wordlength=12 to obtain nucleotide sequences homologous to EBI-3-alt nucleic acid molecules of the invention. BLAST protein searches can be performed with the XBLAST program, score=50, wordlength=3 to obtain amino acid sequences homologous to EBI-3-alt protein molecules of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Alternatively, a PAM 250 residue weight Table, a GAP penalty of 10, and a GAP length penalty of 10 can be used. Another preferred, non-limiting example of a mathematical algorithm utilized for the comparison of sequences is the algorithm of Wilbur-Lipmann which is part of MegAlign™ sequence alignment software. When utilizing the Wilbur-Lipmann algorithm, a K-tuple of 1, a GAP penalty of 3, a window of 5, and diagonals saved set to =5 can be used. Multiple alignment can be performed using the Clustal algorithm.

The invention also provides EBI-3-alt chimeric or fusion proteins. As used herein, an EBI-3-alt “chimeric protein” or “fusion protein” comprises an EBI-3-alt polypeptide operatively linked to a non-EBI-3-alt polypeptide. A “EBI-3-alt polypeptide” refers to a polypeptide having an amino acid sequence corresponding to EBI-3-alt, whereas a “non-EBI-3-alt polypeptide” refers to a polypeptide having an amino acid sequence corresponding to a protein which is not substantially homologous to the EBI-3-alt protein, e.g., a protein which is different from the EBI-3-alt protein and which is derived from the same or a different organism. Within an EBI-3-alt fusion protein the EBI-3-alt polypeptide can correspond to all or a portion of an EBI-3-alt protein. In a preferred embodiment, an EBI-3-alt fusion protein comprises at least one biologically active portion of an EBI-3-alt protein. In another preferred embodiment, an EBI-3-alt fusion protein comprises at least two biologically active portions of an EBI-3-alt protein. In another preferred embodiment, an EBI-3-alt fusion protein comprises at least three biologically active portions of an EBI-3-alt protein. Within the fusion protein, the term “operatively linked” is intended to indicate that the EBI-3-alt polypeptide and the non-EBI-3-alt polypeptide are fused in-frame to each other. The non-EBI-3-alt polypeptide can be fused to the N-terminus or C-terminus of the EBI-3-alt polypeptide.

For example, in one embodiment, the fusion protein is a GST-EBI-3-alt fusion protein in which the EBI-3-alt sequences are fused to the C-terminus of the GST sequences. Such fusion proteins can facilitate the purification of recombinant EBI-3-alt.

The present invention also pertains to variants of the EBI-3-alt proteins which function as either EBI-3-alt agonists (mimetics) or as EBI-3-alt antagonists. Variants of the EBI-3-alt protein can be generated by mutagenesis, e.g., discrete point mutation or truncation of the EBI-3-alt protein. An agonist of the EBI-3-alt protein can retain substantially the same, or a subset, of the biological activities of the naturally occurring form of the EBI-3-alt protein. An antagonist of the EBI-3-alt protein can inhibit one or more of the activities of the naturally occurring form of the EBI-3-alt protein by, for example, competitively binding to an EBI-3-alt receptor of EBI-3-alt-binding protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the EBI-3-alt proteins.

In one embodiment, variants of the EBI-3-alt protein which function as either EBI-3-alt agonists (mimetics) or as EBI-3-alt antagonists can be identified by screening combinatorial libraries of mutants, (e.g., truncation mutants) of the EBI-3-alt protein for EBI-3-alt protein agonist or antagonist activity. In one embodiment, a variegated library of EBI-3-alt variants is generated by combinatorial mutagenesis at the nucleic acid level and is encoded by a variegated gene library. A variegated library of EBI-3-alt variants can be produced by, for example, enzymatically ligating a mixture of synthetic oligonucleotides into gene sequences such that a degenerate set of potential EBI-3-alt sequences is expressible as individual polypeptides, or alternatively, as a set of larger fusion proteins (e.g., for phage display) containing the set of EBI-3-alt sequences therein. There are a variety of methods which can be used to produce libraries of potential EBI-3-alt variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential EBI-3-alt sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the EBI-3-alt protein coding sequence can be used to generate a variegated population of EBI-3-alt fragments for screening and subsequent selection of variants of an EBI-3-alt protein. In one embodiment, a library of coding sequence fragments can be generated by treating a double stranded PCR fragment of an EBI-3-alt coding sequence with a nuclease under conditions wherein nicking occurs only about once per molecule, denaturing the double stranded DNA, renaturing the DNA to form double stranded DNA, which can include sense/antisense pairs from different nicked products, removing single stranded portions from reformed duplexes by treatment with SI nuclease, and ligating the resulting fragment library into an expression vector. By this method, an expression library can be derived which encodes N-terminal, C-terminal and internal fragments of various sizes of the EBI-3-alt protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of EBI-3-alt proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify EBI-3-alt variants (Arkin and Yourvan (1992) PNAS 89:7811-7815; Delgrave et al. (1993) Protein Engineering 6(3):327-331).

In one embodiment, cell based assays can be exploited to analyze a variegated EBI-3-alt library. For example, a library of expression vectors can be transfected into a cell line which can secrete EBI-3-alt protein. Supernatants from the transfected cells are then contacted with EBI-3-alt-responsive cells and the effect of the mutation in EBI-3-alt can be detected, e.g., by measuring any of a number of EBI-3-alt-responsive cell responses. Plasmid DNA can then be recovered from the mutant EBI-3-alt-secreting cells which score for inhibition, or alternatively, potentiation of the EBI-3-alt-dependent response, and the individual clones further characterized.

An isolated EBI-3-alt protein, or a portion or fragment thereof, can be used as an immunogen to generate antibodies that bind EBI-3-alt using standard techniques for polyclonal and monoclonal antibody preparation. The full-length EBI-3-alt protein can be used or, alternatively, the invention provides antigenic peptide fragments of EBI-3-alt for use as immunogens. The antigenic peptide of EBI-3-alt comprises at least 8 amino acid residues of the amino acid sequence shown in SEQ ID NO:2 and encompasses an epitope of EBI-3-alt such that an antibody raised against the peptide forms a specific immune complex with EBI-3-alt. Preferably, the antigenic peptide comprises at least 10 amino acid residues, more preferably at least 15 amino acid residues, even more preferably at least 20 amino acid residues, and most preferably at least 30 amino acid residues. Even more preferably, the antigenic peptide comprises at least 5-10, 15, 20, 30, or more amino acid residues (e.g., contiguous residues) from about amino acids 55-192 of SEQ ID NO:2)

A EBI-3-alt immunogen typically is used to prepare antibodies by immunizing a suitable subject, (e.g., rabbit, goat, mouse or other mammal) with the immunogen. An appropriate immunogenic preparation can contain, for example, recombinantly expressed EBI-3-alt protein or a chemically synthesized EBI-3-alt polypeptide. The preparation can further include an adjuvant, such as Freund's complete or incomplete adjuvant, or similar immunostimulatory agent. Immunization of a suitable subject with an immunogenic EBI-3-alt preparation induces a polyclonal anti-EBI-3-alt antibody response.

Accordingly, another aspect of the invention pertains to anti-EBI-3-alt antibodies. The term “antibody” as used herein refers to immunoglobulin molecules and immunologically active portions of immunoglobulin molecules, i.e., molecules that contain an antigen binding site which specifically binds (immunoreacts with) an antigen, such as EBI-3-alt. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind EBI-3-alt. The term “monoclonal antibody” or “monoclonal antibody composition”, as used herein, refers to a population of antibody molecules that contain only one species of an antigen binding site capable of immunoreacting with a particular epitope of EBI-3-alt. A monoclonal antibody composition thus typically displays a single binding affinity for a particular EBI-3-alt protein with which it immunoreacts.

Polyclonal anti-EBI-3-alt antibodies can be prepared as described above by immunizing a suitable subject with an EBI-3-alt immunogen. The anti-EBI-3-alt antibody titer in the immunized subject can be monitored over time by standard techniques, such as with an enzyme linked immunosorbent assay (ELISA) using immobilized EBI-3-alt. If desired, the antibody molecules directed against EBI-3-alt can be isolated from the mammal (e.g., from the blood) and further purified by well known techniques, such as protein A chromatography to obtain the IgG fraction. At an appropriate time after immunization, e.g., when the anti-EBI-3-alt antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an EBI-3-alt immunogen as described above, and the culture supernatants of the resulting hybridoma cells are screened to identify a hybridoma producing a monoclonal antibody that binds EBI-3-alt.

Any of the many well known protocols used for fusing lymphocytes and immortalized cell lines can be applied for the purpose of generating an anti-EBI-3-alt monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind EBI-3-alt, e.g., using a standard ELISA assay.

Alternative to preparing monoclonal antibody-secreting hybridomas, a monoclonal anti-EBI-3-alt antibody can be identified and isolated by screening a recombinant combinatorial immunoglobulin library (e.g., an antibody phage display library) with EBI-3-alt to thereby isolate immunoglobulin library members that bind EBI-3-alt. Kits for generating and screening phage display libraries are commercially available (e.g., the Pharmacia Recombinant Phage Antibody System, Catalog No. 27-9400-01; and the Stratagene SurZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-EBI-3-alt antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-EBI-3-alt antibody (e.g., monoclonal antibody) can be used to isolate EBI-3-alt by standard techniques, such as affinity chromatography or immunoprecipitation. An anti-EBI-3-alt antibody can facilitate the purification of natural EBI-3-alt from cells and of recombinantly produced EBI-3-alt expressed in host cells. Moreover, an anti-EBI-3-alt antibody can be used to detect EBI-3-alt protein (e.g., in a cellular lysate or cell supernatant) in order to evaluate the abundance and pattern of expression of the EBI-3-alt protein. Anti-EBI-3-alt antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding EBI-3-alt (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., EBI-3-alt proteins, mutant forms of EBI-3-alt, fusion proteins, etc.).

The recombinant expression vectors of the invention can be designed for expression of EBI-3′ alt in prokaryotic or eukaryotic cells. For example, EBI-3-alt can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promotors directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in EBI-3-alt activity assays, in EBI-3-alt ligand binding (e.g., direct assays or competitive assays described in detail below), to generate antibodies specific for EBI-3-alt proteins, as examples. In a preferred embodiment, an EBI-3-alt fusion expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gnl). This viral polymerase is supplied by host strains BL21(DE3) or HMS174(DE3) from a resident X prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the EBI-3-alt expression vector is a yeast expression vector. Examples of vectors for expression in yeast S. cerivisae include pYepSec 1 (Baldari, et al., (1987) EMBO J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, Carlsbad, Calif.).

Alternatively, EBI-3-alt can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to EBI-3-alt mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, EBI-3-alt protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding EBI-3-alt or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) EBI-3-alt protein. Accordingly, the invention further provides methods for producing EBI-3-alt protein using the host cells of the invention. In one embodiment, the method comprises culturing the host cell of invention (into which a recombinant expression vector encoding EBI-3-alt has been introduced) in a suitable medium such that EBI-3-alt protein is produced. In another embodiment, the method further comprises isolating EBI-3-alt from the medium or the host cell.

The host cells of the invention can also be used to produce nonhuman transgenic animals. For example, in one embodiment, a host cell of the invention is a fertilized oocyte or an embryonic stem cell into which EBI-3-alt-coding sequences have been introduced. Such host cells can then be used to create non-human transgenic animals in which exogenous EBI-3-alt sequences have been introduced into their genome or homologous recombinant animals in which endogenous EBI-3-alt sequences have been altered. Such animals are useful for studying the function and/or activity of EBI-3-alt and for identifying and/or evaluating modulators of EBI-3-alt activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous EBI-3-alt gene has been altered by homologous recombination between the endogenous gene and an exogenous DNA molecule introduced into a cell of the animal, e.g., an embryonic cell of the animal, prior to development of the animal.

A transgenic animal of the invention can be created by introducing EBI-3-alt-encoding nucleic acid into the male pronuclei of a fertilized oocyte, e.g., by microinjection, retroviral infection, and allowing the oocyte to develop in a pseudopregnant female foster animal. The EBI-3-alt cDNA sequence of SEQ ID NO:1 or SEQ ID NO:3, or the DNA insert of the plasmid deposited with ATCC as Accession Number ______ can be introduced as a transgene into the genome of a non-human animal. Intronic sequences and polyadenylation signals can also be included in the transgene to increase the efficiency of expression of the transgene. A tissue-specific regulatory sequence(s) can be operably linked to the EBI-3-alt transgene to direct expression of EBI-3-alt protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the EBI-3-alt transgene in its genome and/or expression of EBI-3-alt mRNA in tissues or cells of the animals. A transgenic founder animal can then be used to breed additional animals carrying the transgene. Moreover, transgenic animals carrying a transgene encoding EBI-3-alt can further be bred to other transgenic animals carrying other transgenes.

To create a homologous recombinant animal, a vector is prepared which contains at least a portion of an EBI-3-alt gene into which a deletion, addition or substitution has been introduced to thereby alter, e.g., functionally disrupt, the EBI-3-alt gene. The EBI-3-alt gene can be a human gene (e.g., the cDNA of SEQ ID NO:1 or SEQ ID NO:3), but more preferably, is a non-human homologue of a human EBI-3-alt gene. For example, a mouse EBI-3-alt gene can be used to construct a homologous recombination vector suitable for altering an endogenous EBI-3-alt gene in the mouse genome. In a preferred embodiment, the vector is designed such that, upon homologous recombination, the endogenous EBI-3-alt gene is functionally disrupted (i.e., no longer encodes a functional protein; also referred to as a “knock out” vector). Alternatively, the vector can be designed such that, upon homologous recombination, the endogenous EBI-3-alt gene is mutated or otherwise altered but still encodes functional protein (e.g., the upstream regulatory region can be altered to thereby alter the expression of the endogenous EBI-3-alt protein). In the homologous recombination vector, the altered portion of the EBI-3-alt gene is flanked at its 5′ and 3′ ends by additional nucleic acid of the EBI-3-alt gene to allow for homologous recombination to occur between the exogenous EBI-3-alt gene carried by the vector and an endogenous EBI-3-alt gene in an embryonic stem cell. The additional flanking EBI-3-alt nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced EBI-3-alt gene has homologously recombined with the endogenous EBI-3-alt gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT Publication Numbers WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The EBI-3-alt nucleic acid molecules, EBI-3-alt proteins, anti-EBI-3-alt antibodies, and EBI-3-alt modulators (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an EBI-3-alt protein or anti-EBI-3-alt antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) detection assays (e.g., chromosomal mapping, tissue typing, forensic biology), c) predictive medicine (e.g., diagnostic assays, prognostic assays, monitoring clinical trials); and d) methods of treatment (e.g., therapeutic and prophylactic methods as well as such methods in the context of pharmacogenetics). As described herein, an EBI-3-alt protein of the invention has one or more of the following activities: (i) interaction of an EBI-3-alt protein with a non-EBI-3-alt protein molecule (e.g., an EBI-3-alt receptor or an heteromeric multimer partner); (ii) interaction of an EBI-3-alt protein with one or more other EBI-3-alt protein molecules (e.g., a homomeric multimer partner); (iii) complex formation between an EBI-3-alt protein and an EBI-3-alt receptor; (iv) complex formation between an EBI-3-alt protein and non-EBI-3-alt receptor; (v) modulation of signal transduction pathways; (vi) modulation of a cellular response; (vii) modulation of levels of a EBI-3-alt ligand or metabolic product; (viii) modulation of a cellular response to an EBI-3-alt ligand; and (ix) modulation of cellular homeostasis.

Accordingly, an EBI-3-alt protein, EBI-3-alt modulator, or EBI-3-alt receptor modulator can be used in, for example, (1) activation of an EBI-3-alt-dependent signal transduction pathway; (2) modulation of proliferation, development or differentiation of an EBI-3-alt-expressing cell or EBI-3-alt-receptor-expressing cell; (3) modulation of the proliferation, development or differentiation of a non-EBI-3-alt-expressing cell or non-EBI-3-alt-receptor-expressing cell; (4) modulation of the homeostasis of an EBI-3-alt-expressing cell; and (5) modulation of the homeostasis of a non-EBI-3-alt-expressing cell; and (6) modulation of levels of EBI-3-alt ligands or metabolites.

The isolated nucleic acid molecules of the invention can be used, for example, to express EBI-3-alt protein (e.g., via a recombinant expression vector in a host cell in gene therapy applications), to detect EBI-3-alt mRNA (e.g., in a biological sample) or a genetic alteration in an EBI-3-alt gene, and to modulate EBI-3-alt activity, as described further below. In addition, the EBI-3-alt proteins can be used to screen drugs or compounds which modulate the EBI-3-alt activity as well as to treat disorders characterized by insufficient or excessive production of EBI-3-alt protein or production of EBI-3-alt protein forms which have decreased or aberrant activity compared to EBI-3-alt wild type protein (e.g., EBI-3-alt associated disorders). Moreover, soluble forms of the EBI-3-alt protein can be used to bind EBI-3-alt receptors and influence bioavailability of such a receptor's cognate ligand. In addition, the anti-EBI-3-alt antibodies of the invention can be used to detect and isolate EBI-3-alt proteins and modulate EBI-3-alt activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to EBI-3-alt proteins or have a stimulatory or inhibitory effect on, for example, EBI-3-alt expression or EBI-3-alt activity.

In one embodiment, the invention provides assays for screening candidate or test compounds which bind to or modulate the activity of an EBI-3-alt protein or polypeptide or biologically active portion thereof. In another embodiment, the invention provides assays for screening candidate or test compounds which bind to or modulate the activity of an EBI-3-alt receptor.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage ((Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.)).

In one embodiment, an assay is a cell-based assay in which a cell which expresses EBI-3-alt is contacted with a test compound and the ability of the test compound to modulate EBI-3-alt expression and/or activity determined. Determining the ability of the compound to modulate EBI-3-alt expression can be accomplished, for example, by detecting the presence or absence or amount of an EBI-3-alt transcript of protein (e.g., using a probe based on the nucleotide sequences of the present invention or an anti-EBI-3-alt antibody). Determining the ability of the compound to modulate EBI-3-alt activity can be accomplished, for example, by detecting EBI-3-alt activity in cellular supernatants (e.g., contacting a second cell with the supernatants).

In another embodiment, an assay is a cell-based assay in which a cell which expresses an EBI-3-alt receptor on the cell surface is contacted with a test compound and the ability of the test compound to bind to an EBI-3-alt receptor determined. The cell, for example, can be of mammalian origin or a yeast cell. Determining the ability of the test compound to bind to an EBI-3-alt receptor can be accomplished, for example, by coupling the test compound with a radioisotope or enzymatic label such that binding of the test compound to the EBI-3-alt receptor can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with an EBI-3-alt receptor without the labeling of any of the interactants. For example, a microphysiometer can be used to detect the interaction of a test compound with an EBI-3-alt receptor without the labeling of either the test compound or the receptor. McConnell, H. M. et al. (1992) Science 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and receptor.

In a preferred embodiment, the assay comprises contacting a cell which expresses an EBI-3-alt receptor on the cell surface with an EBI-3-alt protein or biologically-active portion thereof, to form an assay mixture, contacting the assay mixture with a test compound, and determining the ability of the test compound to interact with an EBI-3-alt receptor, wherein determining the ability of the test compound to interact with an EBI-3-alt receptor comprises determining the ability of the test compound to preferentially bind to the EBI-3-alt receptor as compared to the ability of EBI-3-alt, or a biologically active portion thereof, to bind to the receptor. Alternatively, the assay can comprise determining the ability of the test compound to modulate a cellular activity of EBI-3-alt and/or an EBI-3-alt receptor.

Determining the ability of the EBI-3-alt protein to bind to or interact with an EBI-3-alt target molecule, or to modulate an EBI-3-alt activity, can be accomplished by one of the methods described above for determining direct binding. In a preferred embodiment, determining the ability of the EBI-3-alt protein to bind to or interact with an EBI-3-alt target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an EBI-3-alt-responsive regulatory element operatively linked to a nucleic acid encoding a detectable marker, e.g., luciferase), or detecting a cellular response, for example, expression of a differentiated phenotype.

In yet another embodiment, an assay of the present invention is a cell-free assay in which an EBI-3-alt protein or biologically active portion thereof is contacted with a test compound and the ability of the test compound to bind to the EBI-3-alt protein or biologically active portion thereof is determined. Binding of the test compound to the EBI-3-alt protein can be determined either directly or indirectly as described above. In a preferred embodiment, the assay includes contacting the EBI-3-alt protein or biologically active portion thereof with a known compound which binds EBI-3-alt to form an assay mixture, contacting the assay mixture with a test compound, and determining the ability of the test compound to interact with an EBI-3-alt protein, wherein determining the ability of the test compound to interact with an EBI-3-alt protein comprises determining the ability of the test compound to preferentially bind to EBI-3-alt or biologically active portion thereof as compared to the known compound.

In another embodiment, the assay is a cell-free assay in which EBI-3-alt protein or biologically active portion thereof is contacted with a test compound and the ability of the test compound to modulate (e.g., stimulate or inhibit) the activity of the EBI-3-alt protein or biologically active portion thereof is determined. Determining the ability of the test compound to modulate the activity of EBI-3-alt can be accomplished, for example, by determining the ability of the EBI-3-alt protein to bind to an EBI-3-alt target molecule (e.g., an EBI-3-alt binding protein) by one of the methods described above for determining direct binding. Determining the ability of the EBI-3-alt protein to bind to an EBI-3-alt target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

In an alternative embodiment, determining the ability of the test compound to modulate the activity of EBI-3-alt can be accomplished by determining the ability of the EBI-3-alt protein to further modulate an EBI-3-alt target molecule. For example, the activity of the target molecule on a receptor can be determined as previously described.

In yet another embodiment, the cell-free assay involves contacting the EBI-3-alt protein or biologically active portion thereof with a known compound which binds EBI-3-alt to form an assay mixture, contacting the assay mixture with a test compound, and determining the ability of the test compound to interact with an EBI-3-alt protein, wherein determining the ability of the test compound to interact with an EBI-3-alt protein comprises determining the ability of the EBI-3-alt protein to preferentially bind to or modulate the activity of an EBI-3-alt target molecule.

The cell-free assays of the present invention are amenable to use of both soluble and/or membrane-bound forms of isolated proteins (e.g. EBI-3-alt proteins or biologically active portions thereof or EBI-3-alt target molecules). In the case of cell-free assays in which a membrane-bound form an isolated protein is used (e.g., an EBI-3-alt target molecule or receptor) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl═N,N-dimethyl-3-ammonio-1-propane sulfonate.

In more than one embodiment of the above assay methods of the present invention, it may be desirable to immobilize either EBI-3-alt or its target molecule to facilitate separation of complexed from uncomplexed forms of one or both of the proteins, as well as to accommodate automation of the assay. Binding of a test compound to EBI-3-alt, or interaction of EBI-3-alt with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/EBI-3-alt fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or EBI-3-alt protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of EBI-3-alt binding or activity determined using standard techniques.

Other techniques for immobilizing proteins on matrices can also be used in the screening assays of the invention. For example, either EBI-3-alt or its target molecule can be immobilized utilizing conjugation of biotin and streptavidin. Biotinylated EBI-3-alt or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with EBI-3-alt or target molecules but which do not interfere with binding of the EBI-3-alt protein to its target molecule can be derivatized to the wells of the plate, and unbound target or EBI-3-alt trapped in the wells by antibody conjugation. Methods for detecting such complexes, in addition to those described above for the GST-immobilized complexes, include immunodetection of complexes using antibodies reactive with the EBI-3-alt or target molecule, as well as enzyme-linked assays which rely on detecting an enzymatic activity associated with the EBI-3-alt or target molecule.

In another embodiment, modulators of EBI-3-alt expression are identified in a method wherein a cell is contacted with a candidate compound and the expression of EBI-3-alt mRNA or protein in the cell is determined. The level of expression of EBI-3-alt mRNA or protein in the presence of the candidate compound is compared to the level of expression of EBI-3-alt mRNA or protein in the absence of the candidate compound. The candidate compound can then be identified as a modulator of EBI-3-alt expression based on this comparison. For example, when expression of EBI-3-alt mRNA or protein is greater (statistically significantly greater) in the presence of the candidate compound than in its absence, the candidate compound is identified as a stimulator of EBI-3-alt mRNA or protein expression. Alternatively, when expression of EBI-3-alt mRNA or protein is less (statistically significantly less) in the presence of the candidate compound than in its absence, the candidate compound is identified as an inhibitor of EBI-3-alt mRNA or protein expression. The level of EBI-3-alt mRNA or protein expression in the cells can be determined by methods described herein for detecting EBI-3-alt mRNA or protein.

In yet another aspect of the invention, the EBI-3-alt proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with EBI-3-alt (“EBI-3-alt-binding proteins” or “EBI-3-alt-bp”) and modulate EBI-3-alt activity. Such EBI-3-alt-binding proteins are also likely to be involved in the propagation of signals by the EBI-3-alt proteins as, for example, downstream elements of an EBI-3-alt-mediated signaling pathway. Alternatively, such EBI-3-alt-binding proteins are likely to be cell-surface molecules associated with non-EBI-3-alt expressing cells, wherein such EBI-3-alt-binding proteins are involved in secondary cytokine production.

The two-hybrid system is based on the modular nature of most transcription factors, which consist of separable DNA-binding and activation domains. Briefly, the assay utilizes two different DNA constructs. In one construct, the gene that codes for EBI-3-alt is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an EBI-3-alt-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with EBI-3-alt.

This invention further pertains to novel agents identified by the above-described screening assays. Accordingly, it is within the scope of this invention to further use an agent identified as described herein in an appropriate animal model. For example, an agent identified as described herein (e.g., an EBI-3-alt modulating agent, an antisense EBI-3-alt nucleic acid molecule, an EBI-3-alt-specific antibody, or an EBI-3-alt binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the EBI-3-alt, sequences, described herein, can be used to map the location of the EBI-3-alt genes, respectively, on a chromosome. The mapping of the EBI-3-alt sequences to chromosomes is an important first step in correlating these sequences with genes associated with disease.

Briefly, EBI-3-alt genes can be mapped to chromosomes by preparing PCR primers (preferably 15-25 bp in length) from the EBI-3-alt sequences. Computer analysis of the EBI-3-alt sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the EBI-3-alt sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the EBI-3-alt sequences to design oligonucleotide primers, sublocalization can be achieved with panels of fragments from specific chromosomes. Other mapping strategies which can similarly be used to map a EBI-3-alt sequence to its chromosome include in situ hybridization (described in Fan, Y. et al. (1990) PNAS, 87:6223-27), pre-screening with labeled flow-sorted chromosomes, pre-selection by hybridization to chromosome specific cDNA libraries, and search analyses of genomic databases (e.g., FASTA searches of GenBank).

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the EBI-3-alt gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The EBI-3-alt sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the EBI-3-alt sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The EBI-3-alt sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals.

If a panel of reagents from EBI-3-alt sequences described herein is used to generate a unique identification database for an individual, those same reagents can later be used to identify tissue from that individual. Using the unique identification database, positive identification of the individual, living or dead, can be made from extremely small tissue samples.

3. Use of Partial EBI-3-alt Sequences in Forensic Biology

DNA-based identification techniques can also be used in forensic biology.

Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the EBI-3-alt sequences or portions thereof, e.g., fragments derived from the noncoding regions of SEQ ID NO:1 having a length of at least 20 bases, preferably at least 30 bases.

The EBI-3-alt sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such EBI-3-alt probes can be used to identify tissue by species and/or by organ type.

In a similar fashion, these reagents, e.g., EBI-3-alt primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining EBI-3-alt protein and/or nucleic acid expression as well as EBI-3-alt activity, in the context of a biological sample (e.g., blood, serum, cells, tissue) to thereby determine whether an individual is afflicted with a disease or disorder, or is at risk of developing a disorder, associated with aberrant EBI-3-alt expression or activity. The invention also provides for prognostic (or predictive) assays for determining whether an individual is at risk of developing a disorder associated with EBI-3-alt protein, nucleic acid expression or activity. For example, mutations in an EBI-3-alt gene can be assayed in a biological sample. Such assays can be used for prognostic or predictive purpose to thereby prophylactically treat an individual prior to the onset of a disorder characterized by or associated with EBI-3-alt protein, nucleic acid expression or activity.

Another aspect of the invention pertains to monitoring the influence of agents (e.g., drugs, compounds and the like) on the expression or activity of EBI-3-alt in clinical trials.

These and other agents are described in further detail in the following sections.

1. Diagnostic Assays

An exemplary method for detecting the presence or absence of EBI-3-alt in a biological sample involves obtaining a biological sample from a test subject and contacting the biological sample with a compound or an agent capable of detecting EBI-3-alt protein or nucleic acid (e.g., mRNA, genomic DNA) that encodes EBI-3-alt protein such that the presence of EBI-3-alt is detected in the biological sample. A preferred agent for detecting EBI-3-alt mRNA or genomic DNA is a labeled nucleic acid probe capable of hybridizing to EBI-3-alt mRNA or genomic DNA. The nucleic acid probe can be, for example, a full-length EBI-3-alt nucleic acid, such as the nucleic acid of SEQ ID NO:1 or SEQ ID NO:3, the DNA insert of the plasmid deposited with ATCC as Accession Number ______, or a portion thereof, such as an oligonucleotide of at least 15, 30, 50, 100, 250 or 500 nucleotides in length and sufficient to specifically hybridize under stringent conditions to EBI-3-alt mRNA or genomic DNA. Other suitable probes for use in the diagnostic assays of the invention are described herein.

A preferred agent for detecting EBI-3-alt protein is an antibody capable of binding to EBI-3-alt protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect EBI-3-alt mRNA, protein, or genomic DNA in a biological sample in vitro as well as in vivo. For example, in vitro techniques for detection of EBI-3-alt mRNA include Northern hybridizations and in situ hybridizations. In vitro techniques for detection of EBI-3-alt protein include enzyme linked immunosorbent assays (ELISAs), Western blots, immunoprecipitations and immunofluorescence. In vitro techniques for detection of EBI-3-alt genomic DNA include Southern hybridizations. Furthermore, in vivo techniques for detection of EBI-3-alt protein include introducing into a subject a labeled anti-EBI-3-alt antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting EBI-3-alt protein, mRNA, or genomic DNA, such that the presence of EBI-3-alt protein, mRNA or genomic DNA is detected in the biological sample, and comparing the presence of EBI-3-alt protein, mRNA or genomic DNA in the control sample with the presence of EBI-3-alt protein, mRNA or genomic DNA in the test sample.

The invention also encompasses kits for detecting the presence of EBI-3-alt in a biological sample. For example, the kit can comprise a labeled compound or agent capable of detecting EBI-3-alt protein or mRNA in a biological sample; means for determining the amount of EBI-3-alt in the sample; and means for comparing the amount of EBI-3-alt in the sample with a standard. The compound or agent can be packaged in a suitable container. The kit can further comprise instructions for using the kit to detect EBI-3-alt protein or nucleic acid.

2. Prognostic Assays

The diagnostic methods described herein can furthermore be utilized to identify subjects having or at risk of developing a disease or disorder associated with aberrant EBI-3-alt expression or activity. For example, the assays described herein, such as the preceding diagnostic assays or the following assays, can be utilized to identify a subject having or at risk of developing a disorder associated with EBI-3-alt protein, nucleic acid expression or activity such as a EBI-3-alt associated disorder. Thus, the present invention provides a method for identifying a disease or disorder associated with aberrant EBI-3-alt expression or activity in which a test sample is obtained from a subject and EBI-3-alt protein or nucleic acid (e.g, mRNA, genomic DNA) is detected, wherein the presence of EBI-3-alt protein or nucleic acid is diagnostic for a subject having or at risk of developing a disease or disorder associated with aberrant EBI-3-alt expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant EBI-3-alt expression or activity. For example, such methods can be used to determine whether a subject can be effectively treated with an agent for a disorder, such as an EBI-3-alt associated disorder. Thus, the present invention provides methods for determining whether a subject can be effectively treated with an agent for a disorder associated with aberrant EBI-3-alt expression or activity in which a test sample is obtained and EBI-3-alt protein or nucleic acid is detected (e.g., wherein the presence of EBI-3-alt protein or nucleic acid is diagnostic for a subject that can be administered the agent to treat a disorder associated with aberrant EBI-3-alt expression or activity.)

The methods of the invention can also be used to detect genetic alterations in an EBI-3-alt gene, thereby determining if a subject with the altered gene is at risk for a disorder characterized by aberrant EBI-3-alt activity (e.g., an EBI-3-alt associated disorder). In preferred embodiments, the methods include detecting, in a sample of cells from the subject, the presence or absence of a genetic alteration characterized by at least one of an alteration affecting the integrity of a gene encoding an EBI-3-alt protein, or the mis-expression of the EBI-3-alt gene. For example, such genetic alterations can be detected by ascertaining the existence of at least one of 1) a deletion of one or more nucleotides from an EBI-3-alt gene; 2) an addition of one or more nucleotides to an EBI-3-alt gene; 3) a substitution of one or more nucleotides of an EBI-3-alt gene, 4) a chromosomal rearrangement of an EBI-3-alt gene; 5) an alteration in the level of a messenger RNA transcript of an EBI-3-alt gene, 6) aberrant modification of an EBI-3-alt gene, such as of the methylation pattern of the genomic DNA, 7) the presence of a non-wild type splicing pattern of a messenger RNA transcript of an EBI-3-alt gene, 8) a non-wild type level of an EBI-3-alt protein, 9) allelic loss of an EBI-3-alt gene, and 10) inappropriate post-translational modification of an EBI-3-alt protein. As described herein, there are a large number of assay techniques known in the art which can be used for detecting alterations in an EBI-3-alt gene. A preferred biological sample is serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the EBI-3-alt gene (see Abravaya et al. (1995) Nucleic Acids Res .23:675-682). This method can include the steps of collecting a sample of cells from a patient, isolating nucleic acid (e.g., genomic, mRNA or both) from the cells of the sample, contacting the nucleic acid sample with one or more primers which specifically hybridize to an EBI-3-alt gene under conditions such that hybridization and amplification of the EBI-3-alt gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J.C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et all, 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an EBI-3-alt gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicate mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in EBI-3-alt can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in EBI-3-alt can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the EBI-3-alt gene and detect mutations by comparing the sequence of the sample EBI-3-alt with the corresponding wild-type (control) sequence. Examples of sequencing reactions include those based on techniques developed by Maxim and Gilbert ((1977) PNAS 74:560) or Sanger ((1977) PNAS 74:5463). It is also contemplated that any of a variety of automated sequencing procedures can be utilized when performing the diagnostic assays (Naeve et al (1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the EBI-3-alt gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type EBI-3-alt sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in EBI-3-alt cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an EBI-3-alt sequence, e.g., a wild-type EBI-3-alt sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in EBI-3-alt genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat Res 285:125-144; and Hayashi (1992) Genet Anal Tech Appl 9:73-79). Single-stranded DNA fragments of sample and control EBI-3-alt nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an EBI-3-alt gene.

Furthermore, any cell type or tissue in which EBI-3-alt is expressed may be utilized in the prognostic assays described herein.

3. Monitoring of Effects During Clinical Trials

Monitoring the influence of agents (e.g., drugs, compounds) on the expression or activity of EBI-3-alt (e.g., activation of an EBI-3-alt-dependent signal transduction pathway, modulation of an EBI-3-alt associated activity, modulation of an EBI-3-alt associated disorder) can be applied not only in basic drug screening, but also in clinical trials. For example, the effectiveness of an agent determined by a screening assay as described herein to increase EBI-3-alt gene expression, protein levels, or upregulate EBI-3-alt activity, can be monitored in clinical trials of subjects exhibiting decreased EBI-3-alt gene expression, protein levels, or downregulated EBI-3-alt activity. Alternatively, the effectiveness of an agent determined by a screening assay to decrease EBI-3-alt gene expression, protein levels, or downregulate EBI-3-alt activity, can be monitored in clinical trials of subjects exhibiting increased EBI-3-alt gene expression, protein levels, or upregulated EBI-3-alt activity. In such clinical trials, the expression or activity of EBI-3-alt and, preferably, other genes that have been implicated in, for example, an EBI-3-alt associated disorder can be used as a “read out” or markers of the level that a particular cell is affected by the disorder.

For example, and not by way of limitation, genes, including EBI-3-alt, that are modulated in cells by treatment with an agent (e.g., compound, drug or small molecule) which modulates EBI-3-alt activity (e.g., identified in a screening assay as described herein) can be identified. Thus, to study the effect of agents on EBI-3-alt associated disorders, for example, in a clinical trial, cells can be isolated and RNA prepared and analyzed for the levels of expression of EBI-3-alt and other genes implicated in the EBI-3-alt associated disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of EBI-3-alt or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of an EBI-3-alt protein, mRNA, or genomic DNA in the preadministration sample; (iii) obtaining one or more post-administration samples from the subject; (iv) detecting the level of expression or activity of the EBI-3-alt protein, mRNA, or genomic DNA in the post-administration samples; (v) comparing the level of expression or activity of the EBI-3-alt protein, mRNA, or genomic DNA in the pre-administration sample with the EBI-3-alt protein, mRNA, or genomic DNA in the post administration sample or samples; and (vi) altering the administration of the agent to the subject accordingly. For example, increased administration of the agent may be desirable to increase the expression or activity of EBI-3-alt to higher levels than detected, i.e., to increase the effectiveness of the agent. Alternatively, decreased administration of the agent may be desirable to decrease expression or activity of EBI-3-alt to lower levels than detected, i.e. to decrease the effectiveness of the agent. According to such an embodiment, EBI-3-alt expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant EBI-3-alt expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the EBI-3-alt molecules of the present invention or EBI-3-alt modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant EBI-3-alt expression or activity, by administering to the subject an agent which modulates EBI-3-alt expression or at least one EBI-3-alt activity. Subjects at risk for a disease which is caused or contributed to by aberrant EBI-3-alt expression or activity can be identified by, for example, any or a combination of diagnostic or prognostic assays as described herein. Administration of a prophylactic agent can occur prior to the manifestation of symptoms characteristic of the EBI-3-alt aberrancy, such that a disease or disorder is prevented or, alternatively, delayed in its progression. Depending on the type of EBI-3-alt aberrancy, for example, an EBI-3-alt agonist or EBI-3-alt antagonist agent can be used for treating the subject. The appropriate agent can be determined based on screening assays described herein.

2. Therapeutic Methods

Another aspect of the invention pertains to methods of modulating EBI-3-alt expression or activity for therapeutic purposes. The modulatory method of the invention involves contacting a cell with an agent that modulates one or more of the activities of EBI-3-alt protein activity associated with the cell. An agent that modulates EBI-3-alt protein activity can be an agent as described herein, such as a nucleic acid or a protein, a naturally-occurring target molecule of an EBI-3-alt protein, a peptide, an EBI-3-alt peptidomimetic, or other small molecule. In one embodiment, the agent stimulates one or more EBI-3-alt protein activity. Examples of such stimulatory agents include active EBI-3-alt protein and a nucleic acid molecule encoding EBI-3-alt that has been introduced into the cell. In another embodiment, the agent inhibits one or more EBI-3-alt protein activity. Examples of such inhibitory agents include antisense EBI-3-alt nucleic acid molecules and anti-EBI-3-alt antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of an EBI-3-alt protein or nucleic acid molecule. In one embodiment, the method involves administering an agent (e.g., an agent identified by a screening assay described herein), or combination of agents that modulates (e.g., upregulates or downregulates) EBI-3-alt expression or activity. In another embodiment, the method involves administering an EBI-3-alt protein or nucleic acid molecule as therapy to compensate for reduced or aberrant EBI-3-alt expression or activity.

A preferred embodiment of the present invention involves a method for treatment of a disease or disorder associated with an EBI-3-alt protein which includes the step of administering a therapeutically effective amount of an antibody to an EBI-3-alt protein to a subject. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays as described herein.

Stimulation of EBI-3-alt activity is desirable in situations in which EBI-3-alt is abnormally downregulated and/or in which increased EBI-3-alt activity is likely to have a beneficial effect. One example of such a situation is where a subject has a disorder characterized by aberrant EBI-3-alt activity (e.g., an EBI-3-alt associated disorder).

3. Pharmacogenomics

The EBI-3-alt molecules of the present invention, as well as agents, or modulators which have a stimulatory or inhibitory effect on EBI-3-alt activity (e.g., EBI-3-alt gene expression) as identified by a screening assay described herein can be administered to individuals to treat (prophylactically or therapeutically) disorders (e.g, EBI-3-alt associated disorders) associated with aberrant EBI-3-alt activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an EBI-3-alt molecule or EBI-3-alt modulator as well as tailoring the dosage and/or therapeutic regimen of treatment with an EBI-3-alt molecule or EBI-3-alt modulator.

Pharmacogenomics deals with clinically significant hereditary variations in the response to drugs due to altered drug disposition and abnormal action in affected persons. See e.g., Eichelbaum, M., Clin Exp Pharmacol Physiol, 1996, 23(10-11):983-985 and Linder, M. W., Clin Chem, 1997, 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., an EBI-3-alt protein or EBI-3-alt receptor of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme is the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., an EBI-3-alt molecule or EBI-3-alt modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with an EBI-3-alt molecule or EBI-3-alt modulator, such as a modulator identified by one of the exemplary screening assays described herein.

This invention is further illustrated by the following examples which should not be construed as limiting. The contents of all references, patents and published patent applications cited throughout this application are hereby incorporated by reference.

### EXAMPLES

### Example 1

### Isolation And Characterization of Human EBI-3-alt cDNAs

Isolation of Human EBI-3-alt

Human placental RNA was acquired from a commercial source (Ambion). Poly A-plus mRNA was isolated using the Promega PolyA Tract Isolation kit following the manufacturer's procedure. A commercially available kit was used to generate cDNA from the placental mRNA following manufacturer's procedure (Gibco-BRL). EcoRI adaptors were ligated onto the synthesized cDNA, then size selected on a 1.5% agarose gel. Nucleic acids of 2.5 kb to 9.0 kb in size were selected and extracted from the agarose. These selected cDNAs were ligated into the EcoRI site of the pCRII vector (Invitrogen). One half of the constructed library was transferred (transformation by electroporation) into the XL1 Blue (Stratagene) strain of E. Coli and plated onto ten agar-medium plates (ø15 cm) containing ampicillin (100 ug/ml). The plates were allowed to incubate overnight at 37° C., then duplicate filters were lifted from each plate. The filters were denatured, neutralized, and then allowed to air dry before baking for 2 hours at 80° C. The baked filters were then probed using a ³²P-labeled DNA probe corresponding to the open reading frame of human EBI-3 (generated by PCR). Hybridization was carried out over night at 42° C. in hybridization buffer (6× SSC, 1× Denhardt's solution, 0.05% NaP₄O₇). The filters were then washed three times in 0.2× SSC, 0.1% SDS at 65° C. The washed filters were air dried and then exposed to radiography film. Two positive colonies were selected and re-plated at different dilutions. The plates which gave well-isolated colonies (e.g., were appropriately diluted) were probed again using the same probe and procedure described above. Two isolated positive colonies were selected and grown overnight at 37° C. in 5ml of LB broth. DNA was prepared from 1.5 ml of each culture. A portion of the DNA was digested with EcoRI and analyzed by electrophoresis in agarose. The clones were reconfirmed with PCR using primers corresponding to sequences within the EBI-3 open reading frame. These two clones were then sequenced using a fluorescent-based dye terminator PCR amplification system and an ABI Prism 373 sequencer.

Characterization of Human EBI-3-alt

The nucleic acid sequence of human EBI-3-alt (SEQ ID NO:1) was used to search a nucleotide database (GenBank) using a FASTA package (see, e.g., Pearson (1994) Methods Mol. Biol.24:307-331). This search revealed the mRNA of the human cytokine receptor (EBI-3; GenBank Acc. No. L08187) is 99.3% identical to the cDNA of human EBI-3-alt from nucleotides 170-860.

The amino acid sequence of human EBI-3-alt (SEQ ID NO:2) was used to search a translated DNA database (GenBank) using a TFASTXY package (see, e.g., Pearson et al (1997) Genomics 46:24-36). This search revealed that the human cytokine receptor (EBI-3; translated sequence of GenBank Acc. No. L08187) and a portion of H. sapiens chromosome 19 (translated sequence of GenBank Acc. No. AC005578) are 100% identical to EBI-3-alt from residues 1-35, and from residues 1-21, respectively. In addition, this search revealed that the nitrous oxide reductase catalytic subunit of Pseudomonas fluorescens (translated sequence of GenBank Acc. No. AF056319) is 50% identical to EBI-3-alt from residue 5 to residue 32. An alignment of the human EBI-3 and the human EBI-3-alt is shown in FIG. 4.

The amino acid sequence of human EBI-3-alt (SEQ ID NO:2) was used to search a database of protein domains and signature sequences. This search resulted in the identification of a “receptor_cytokines_{—}1” signature sequence at residues 35-48. This sequence in the EBI-3-alt protein contains a pair of cysteine residues that are conserved in the receptor_cytokines_{—}1 signature sequence (cys 35 and cys 46 of SEQ ID NO:2).

Structural analysis of the amino acid sequence of the human EBI-3-alt protein (SEQ ID NO:2) revealed that the primary sequence contains an N-terminal EBI-3-like domain (residues 1-54 of SEQ ID NO:2), and a C-terminal unique domain (residues 55-192 of SEQ ID NO:2).

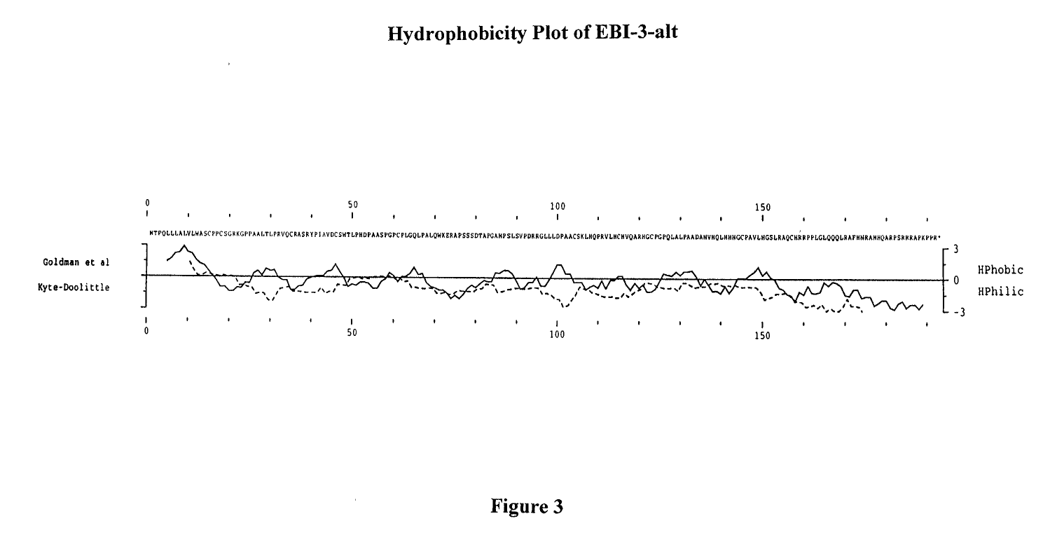

A hydrophobicity analysis was carried out on the protein sequence of EBI-3-alt. The results of this analysis are shown in FIG. 3.

### Example 2

### Expression of Recombinant EBI-3-alt Protein in Bacterial Cells

EBI-3-alt can be expressed as a recombinant glutathione-S-transferase (GST) fusion polypeptide in E. coli and the fusion polypeptide can be isolated and characterized. Specifically, EBI-3-alt is fused to GST and this fusion polypeptide is expressed in E. coli, e.g., strain PEB199. Expression of the GST-EBI-3-alt fusion protein in PEB 199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant EBI-3-alt Protein in COS Cells

To express the EBI-3-alt gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire EBI-3-alt protein and a HA tag (Wilson et al. (1984) Cell 37:767) fused in-frame to its 3′ end of the fragment is cloned into the polylinker region of the vector, thereby placing the expression of the recombinant protein under the control of the CMV promoter.

To construct the plasmid, the EBI-3-alt DNA sequence is amplified by PCR using two primers. The 5′ primer contains the restriction site of interest followed by approximately twenty nucleotides of the EBI-3-alt coding sequence starting from the initiation codon; the 3′ end sequence contains complementary sequences to the other restriction site of interest, a translation stop codon, the HA tag and the last 20 nucleotides of the EBI-3-alt coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the EBI-3-alt gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the EBI-3-alt-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989. The expression of the EBI-3-alt protein is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated proteins are then analyzed by SDS-PAGE.

Alternatively, DNA containing the EBI-3-alt coding sequence is cloned directly into the polylinker of the pCDNA/Amp vector using the appropriate restriction sites. The resulting plasmid is transfected into COS cells in the manner described above, and the expression of the EBI-3-alt protein is detected by radiolabelling and immunoprecipitation using an EBI-3-alt specific monoclonal antibody

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated nucleic acid molecule selected from the group consisting of:
a) a nucleic acid molecule comprising a nucleotide sequence which is at least 75% homologous to a nucleotide sequence of SEQ ID NO:1, SEQ ID NO:3, the DNA insert of the plasmid deposited with ATCC as Accession Number ______, or a complement thereof,
b) a nucleic acid molecule comprising a fragment of at least 30 nucleotides of a nucleic acid comprising the nucleotide sequence of SEQ ID NO:1, SEQ ID NO:3, the DNA insert of the plasmid deposited with ATCC as Accession Number ______, or a complement thereof;
c) a nucleic acid molecule which encodes a polypeptide comprising an amino acid sequence at least about 60% homologous to the amino acid sequence of SEQ ID NO:2, or an amino acid sequence encoded by the DNA insert of the plasmid deposited with ATCC as Accession Number ______;
d) a nucleic acid molecule which encodes a fragment of a polypeptide comprising the amino acid sequence of SEQ ID NO:2, or the polypeptide encoded by the DNA insert of the plasmid deposited with ATCC as Accession Number ______, wherein the fragment comprises at least 10 contiguous amino acid residues of the amino acid sequence of SEQ ID NO:2, or the polypeptide encoded by the DNA insert of the plasmid deposited with ATCC as Accession Number ______, and
e) a nucleic acid molecule which encodes a naturally occurring allelic variant of a polypeptide comprising the amino acid sequence of SEQ ID NO:2, or an amino acid sequence encoded by the DNA insert of the plasmid deposited with ATCC as Accession Number ______, wherein the nucleic acid molecule hybridizes to a complement of a nucleic acid molecule comprising SEQ ID NO:1 or SEQ ID NO:3 under stringent conditions.

2. The isolated nucleic acid molecule of claim 1 which is selected from the group consisting of:
a) a nucleic acid molecule comprising the nucleotide sequence of SEQ ID NO:1, SEQ ID NO:3, or the DNA insert of the plasmid deposited with ATCC as Accession Number ______, or a complement thereof; and
b) a nucleic acid molecule which encodes a polypeptide comprising the amino acid sequence of SEQ ID NO:2, or an amino acid sequence encoded by the DNA insert of the plasmid deposited with ATCC as Accession Number ______.

3. The nucleic acid molecule of claim 1 further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of SEQ ID NO:2, or the polypeptide encoded by the DNA insert of the plasmid deposited with ATCC as Accession Number ______, wherein the fragment comprises at least 10 contiguous amino acids of SEQ ID NO:2, or the amino acid sequence encoded by the DNA insert of the plasmid deposited with ATCC as Accession Number ______;
b) a naturally occurring allelic variant of a polypeptide comprising the amino acid sequence of SEQ ID NO:2, or an amino acid sequence encoded by the DNA insert of the plasmid deposited with ATCC as Accession Number ______, wherein the polypeptide is encoded by a nucleic acid molecule which hybridizes to a complement of a nucleic acid molecule comprising SEQ ID NO:1 or SEQ ID NO:3, under stringent conditions; and
c) a polypeptide which is encoded by a nucleic acid molecule comprising a nucleotide sequence which is at least 75% homologous to a nucleic acid comprising the nucleotide sequence of SEQ ID NO:1 or SEQ ID NO:3, or the DNA insert of the plasmid deposited with ATCC as Accession Number ______.
d) a polypeptide comprising an amino acid sequence which is at least 60% homologous to the amino acid sequence of SEQ ID NO:2, or the polypeptide encoded by the DNA insert of the plasmid deposited with ATCC as Accession Number ______.

9. The isolated polypeptide of claim 8 comprising the amino acid sequence of SEQ ID NO:2, or an amino acid sequence encoded by the DNA insert of the plasmid deposited with ATCC as Accession Number ______.

10. The polypeptide of claim 8 further comprising heterologous amino acid sequences.

11. An antibody which selectively binds to a polypeptide of claim 8.

12. A method for producing a polypeptide selected from the group consisting of:
a) a polypeptide comprising the amino acid sequence of SEQ ID NO:2, or an amino acid sequence encoded by the DNA insert of the plasmid deposited with ATCC as Accession Number ______;
b) a fragment of a polypeptide comprising the amino acid sequence of SEQ ID NO:2, or an amino acid sequence encoded by the DNA insert of the plasmid deposited with ATCC as Accession Number ______ wherein the fragment comprises at least 10 contiguous amino acids of SEQ ID NO:2, or the amino acid sequence encoded by the DNA insert of the plasmid deposited with ATCC as Accession Number ______; and
c) a naturally occurring allelic variant of a polypeptide comprising the amino acid sequence of SEQ ID NO:2, or an amino acid sequence encoded by the DNA insert of the plasmid deposited with ATCC as Accession Number ______, wherein the polypeptide is encoded by a nucleic acid molecule which hybridizes to a complement of a nucleic acid molecule comprising SEQ ID NO:1 or SEQ ID NO:3, under stringent conditions;

13. A method for detecting the presence of a polypeptide of claim 8 in a sample comprising:
a) contacting the sample with a compound which selectively binds to the polypeptide; and
b) determining whether the compound binds to the polypeptide in the sample to thereby detect the presence of a polypeptide of claim 8 in the sample.

14. The method of claim 13, wherein the compound which binds to the polypeptide is an antibody.

15. A kit comprising a compound which selectively binds to a polypeptide of claim 8 and instructions for use.

16. A method for detecting the presence of a nucleic acid molecule in claim 1 in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of a nucleic acid molecule of claim 1 in the sample.

17. The method of claim 16, wherein the sample comprises mRNA molecules and is contacted with a nucleic acid probe.

18. A kit comprising a compound which selectively hybridizes to a nucleic acid molecule of claim 1 and instructions for use.

19. A method for identifying a compound which binds to a polypeptide of claim 8 comprising:
a) contacting the polypeptide, or a cell expressing the polypeptide with a test compound; and
b) determining whether the polypeptide binds to the test compound.

20. The method of claim 19, wherein the binding of the test compound to the polypeptide is detected by a method selected from the group consisting of:
a) detection of binding by direct detection of test compound/polypeptide binding;
b) detection of binding using a competition binding assay; and
c) detection of binding using an assay for EBI-3-alt activity.

21. A method of modulating the activity of a polypeptide of claim 8 comprising contacting the polypeptide or a cell expressing the polypeptide with a compound which binds to the polypeptide in a sufficient concentration to modulate the activity of the polypeptide.

22. A method for identifying a compound which modulates the activity of a polypeptide of claim 8 comprising:
a) contacting a polypeptide of claim 8 with a test compound; and
b) determining the effect of the test compound on the activity of the polypeptide to thereby identify a compound which modulates the activity of the polypeptide.

23. A method of for identifying a compound which modulates a EBI-3-alt associated disorder comprising:
a) contacting a cell which expresses an EBI-3-alt receptor with a compound to form an assay mixture:
b) contacting the assay mixture with EBI-3-alt;
c) determining whether the compound modulates EBI-3-alt activity, such that a modulatory compound is identified.
